(19) United States
(12) Patent Application Publication
(10) Pub. No.: US 2026/0193587 A1
(43) Pub. Date: Jul. 9, 2026

(54) SPECTRAL ENCODING OF TISSUE BEHAVIOR

(71) Applicant: VALO HEALTH, INC., Lexington, MA (US)

(72) Inventors: Nathan Bays, Boston, MA (US); Brian Schriver, Boston, MA (US)

(21) Appl. No.: 19/133,787

(22) PCT Filed: Nov. 27, 2023
(86) PCT No.: PCT/US2023/081085

§ 371 Date: May 29, 2025

Related U.S. Application Data
(60) Provisional application No. 63/429,797, filed on Dec. 2, 2022.

(51) Int. Cl. C12M 1/34; C12M 1/36; C12M 3/00

(52) U.S. Cl. CPC C12M 41/46; C12M 21/08; C12M 41/48

(57) ABSTRACT

Systems, methods, and non-transitory computer readable medium are disclosed for encoding tissue behavior. A bioreactor may be implemented, where the bioreactor comprises a device configured for growing tissue and a sensor assembly configured to detect functional response(s) of a tissue within the device. The systems and methods may also include a control unit communicatively coupled to the bioreactor and configured to obtain, from the bioreactor, waveform(s) comprising functional response(s) of the tissue within the device over a time frame. The control unit may also be configured to transform the one or more waveforms to spectral representation(s) that provide frequency-based characterizations of the functional response(s) of the tissue during the time frame thereby encoding a behavior of the tissue.

### RELATED APPLICATIONS

This application claims the benefit of U.S. Provisional Application No. 63/429,797 (filed on Dec. 2, 2022), which is incorporated by reference herein in its entirety.

### TECHNICAL FIELD

The present disclosure relates to encoding the behavior of an engineered tissue. Particularly, but not exclusively, the present disclosure relates to using a spectral representation to encode the behavior of an engineered tissue. Particularly, but not exclusively, the present disclosure relates to encoding the behavior of an engineered muscle tissue using a spectral representation of a functional response of the engineered muscle tissue.

### BACKGROUND

Tissue behavior can be measured and modeled as a waveform of tissue response versus time. The resulting waveform is a fundamental and valuable unit of representation and encoding for tissue data. Here, encoding means a numerical representation (vectorization) of tissue response versus time and can be performed for many different conditions, types of tissue response, and measurement systems. This representation and encoding of tissue behavior as waveforms provides a rich substrate for downstream analysis. However, existing approaches to featurization of such waveforms require the extraction of specific features from peaks within the waveform. These features are based on an a priori expectation of the behavior of the tissue. For instance, in measuring cardiomyocyte contraction, existing methods expect contractile peaks of certain shape and regularity. Moreover, feature vectors obtained from tissue waveforms using existing methods may not be suitable for comparison due to factors such as differences in vector length and differences due to the waveforms being obtained from different sources (e.g., different tissue types, experimental protocols, bioreactors, etc.).

Therefore, there is a need for new approaches to encoding tissue behavior which do not require a priori expectation of tissue behavior and which allow for direct, and efficient, comparison.

### SUMMARY OF DISCLOSURE

According to an aspect of the present disclosure there is provided a system for encoding tissue behavior. The system comprises a bioreactor and a control unit communicatively coupled to the bioreactor. The bioreactor comprises a device configured for growing tissue and a sensor assembly configured to detect one or more functional responses of a tissue within the device. The control unit is configured to obtain, from the bioreactor, one or more waveforms comprising one or more functional responses of the tissue within the device over a time frame and transform the one or more waveforms to one or more spectral representations. The one or more spectral representations provide frequency-based characterizations of the one or more functional responses of the tissue during the time frame thereby encoding a behavior of the tissue.

According to a further aspect of the present disclosure there is provided a computer implemented method for encoding tissue behavior. The method comprises obtaining a first waveform comprising a functional response of a first tissue over a first time frame, wherein the first tissue is an engineered tissue, and transforming the first waveform to a first spectral representation thereof, wherein the first spectral representation provides a frequency-based characterization of the functional response of the first tissue during the first time frame thereby encoding a behavior of the first tissue.

According to another aspect of the present disclosure there is provided a computer-implemented method for generating a tissue behavior prediction model. The method comprises obtaining a plurality of waveforms comprising functional responses of a plurality of engineered tissues over time, the plurality of engineered tissues comprising at least a first tissue group associated with a first behavior and a second tissue group associated with a second behavior. The method further comprises transforming the plurality of waveforms to a corresponding plurality of spectral representations, wherein the corresponding plurality of spectral representations provide frequency-based characterizations of the functional responses of the plurality of engineered tissues. The method further comprises generating a prediction model using the corresponding plurality of spectral representations, wherein the prediction model assigns a probability that a tissue exhibits the first behavior or the second behavior based on a spectral representation of the tissue.

According to a further aspect of the present disclosure there is provided a computer implemented method of grouping tissue behaviors. The method comprises obtaining a plurality of waveforms comprising functional responses of a plurality of engineered tissues over time, transforming the plurality of waveforms to a corresponding plurality of spectral representations, and grouping the plurality of engineered tissues into at least two behavioral groups based on the corresponding plurality of spectral representations.

According to a further aspect of the present disclosure there is provided a computer implemented method for mechanism of action identification. The method comprises obtaining a first spectral representation of a first functional response of a first tissue, wherein the first tissue is associated with a first intervention having a known mechanism of action, and obtaining a second spectral representation of a second functional response of a second tissue, wherein the second tissue is associated with a second intervention having an unknown mechanism of action. The first tissue and the second tissue are engineered tissues. The method further comprises comparing the first spectral representation to the second spectral representation and determining a potential mechanism of action for the second intervention based on the comparing.

Further aspects and embodiments of the present disclosure are set out in the appended statements.

In accordance with the above, and with the disclosure herein, the present disclosure includes applying certain features or aspects with or by use of, a particular machine, e.g., a bioreactor. In various aspects, the bioreactor may comprise a device configured for growing tissue (e.g., human body tissue such as muscle tissue, cardiac tissue, and/or skeletal muscle tissue). Additionally, or alternatively, bioreactor may comprise a sensor assembly configured to detect one or more functional responses of the tissue within the device.

Further, the present disclosure includes effecting a transformation or reduction of a particular article to a different state or thing, e.g., the transformation or reduction of functional responses of tissue, e.g., within a bioreactor as sensed by a sensor assembly as waveforms, to a different state or thing, e.g., the generation or creation of corresponding spectral representation(s) that provide frequency-based characterizations of the functional responses of the tissue to encode the behavior of the tissue.

Still further, the present disclosure includes improvements in computer functionality or in improvements to other technologies at least because the disclosure herein discloses an artificial intelligence (AI) based model, e.g., a prediction model, that is trained or otherwise generated with spectral representation data representative of functional responses of tissue (e.g., engineered human tissue). The prediction model, when deployed on the underlying system, allows the systems and methods of the present disclosure to execute with fewer iterations, and use fewer computing resources, than prior art related systems and methods.

That is, the present disclosure describes improvements in the functioning of the computer itself or “any other technology or technical field” because the increased predictive improvement provided by the prediction model allows the underlying computer system to utilize less processing and memory resources compared to prior art systems and methods. This is at least because the prediction model can generate or determine a probability that a given tissue will exhibit a given behavior, without the need for empirical computer simulation across a wide range of tests using multiple compute cycles and data. Therefore, use of the prediction model results in fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials on human tissue that can result in, and require, large database and memory utilization and processor usage to arrive at a similar real-world or simulated results that has a same or similar result. The disclosed systems and methods describe generation and/or use of a bioreactor for growing and testing tissue for defining a limited set of data specific to the tissue (e.g., human engineered tissue), which requires less memory usage and/or processing utilization compared to a conventional approach where large sets of unknown, potentially irrelevant data is used or required.

In addition, the present disclosure relates to improvement to other technologies or technical fields at least because the systems and methods of the present disclosure provide a robust, efficient, and comparable encoding of tissue behavior that can be used to improve the efficiency and performance of several downstream drug discovery and development tasks. This may be performed, for example, by a prediction model that is trained or otherwise generated with spectral representations as training data that defines functional responses of tissue (e.g., engineered human tissue). The prediction model may be deployed on an underlying computing device or system, thereby, improving its accuracy and prediction in performing drug discovery and development tasks as described herein.

Still further, the present disclosure includes specific features other than what is well-understood, routine, conventional activity in the field, and/or otherwise adds unconventional steps that confine the disclosure to a particular useful application, e.g., systems and methods for encoding tissue behavior, generating a tissue behavior prediction model, grouping of tissue behaviors, and/or performing mechanism of action identification, e.g., as related to drug discovery and development tasks.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF DRAWINGS

Embodiments of the invention will now be described, by way of example only, and with reference to the accompanying drawings, in which:

FIG. 1A shows a system for encoding tissue behavior according to an aspect of the present disclosure;

FIG. 1B shows an example protocol carried out by the system of FIG. 1A according to embodiments of the present disclosure;

FIG. 2A shows a waveform comprising a functional response of a tissue according to embodiments of the present disclosure;

FIG. 2B shows a spectral representation of the waveform shown in FIG. 2A according to embodiments of the present disclosure;

FIG. 3A illustrates a process for determining a relative spectral representation according to embodiments of the present disclosure;

FIG. 3B illustrates a further example of a relative spectral representation according to embodiments of the present disclosure;

FIG. 3C illustrates the encoding of abnormal tissue behavior in spectral representations according to embodiments of the present disclosure;

FIG. 4 illustrates grouping of spectral representations according to an aspect of the present disclosure;

FIG. 5 illustrates a prediction model to assign a probable behavior to an engineered tissue according to an aspect of the present disclosure;

FIGS. 6A-C shows a method for encoding tissue behavior according to an aspect of the present disclosure;

FIG. 7 shows a method for determining tissue behavior according to an embodiment of the present disclosure;

FIG. 8 shows a method for grouping of tissue behaviors using spectral representations according to an aspect of the present disclosure;

FIG. 9 shows a method for generating a prediction model according to an aspect of the present disclosure;

FIG. 10 shows a method for mechanism of action identification according to an aspect of the present disclosure;

FIG. 11 shows an example computing system for carrying out the methods of the present disclosure; and

FIGS. 12A-P illustrate an example implementation of the present disclosure.

### DETAILED DESCRIPTION

The systems and methods of the present disclosure provide a robust, efficient, and comparable encoding of tissue behavior which can be used to improve the efficiency and performance of several downstream drug discovery and development tasks.

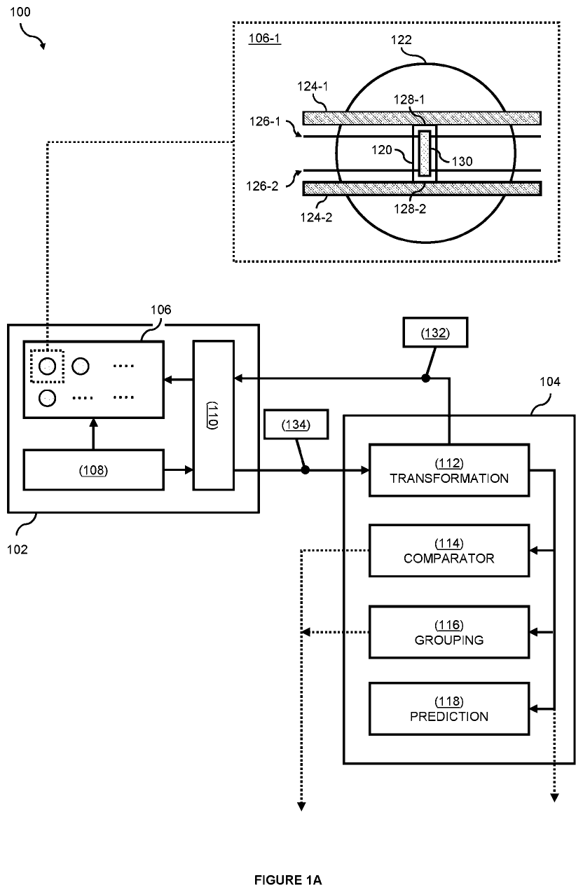

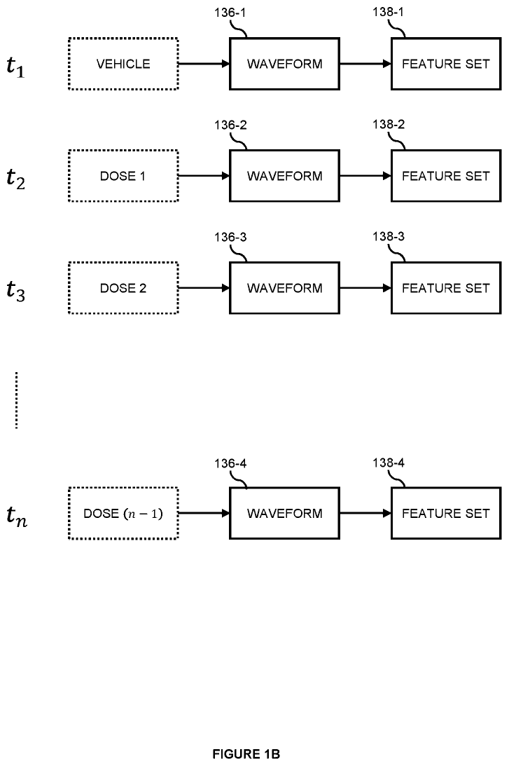

FIG. 1A shows a system 100 for encoding tissue behavior according to an aspect of the present disclosure.

The system 100 comprises a bioreactor 102 and a control unit 104. The bioreactor 102 comprises a device 106 for growing tissue, a sensor assembly 108, and an interface 110. The control unit 104 comprises a transformation module 112, a shift detection module 114, a grouping module 116, and a prediction module 118. The interface 110 communicatively couples the bioreactor 102 and the control unit 104 such that data may be exchanged between the bioreactor 102 and the control unit 104. In various aspects, the bioreactor is used for growing tissue (e.g., tissue 130, such as human body tissue including, by way of non-limiting example, muscle tissue, cardiac tissue, skeletal muscle tissue, or other human tissue). For example, as shown in the expanded portion 106-1 of the device 106, the device 106, or substrate, comprises one or more wells, such as the well 120, one or more cell culture wells, such as the cell culture well 122, a pair of electrodes including a first electrode 124-1 and a second electrode 124-2, and a pair of elements including a first element 126-1 and a second element 126-2. The well 120 is positioned within the cell culture well 122 and has a bottom on the device 106, a first end 128-1, and a second end 128-2. The well 120 is configured for growing a tissue 130 from cells seeded therein. Culture medium may be added to the cell culture well 122 for growing and/or sustaining the tissue 130.

The pair of electrodes are separated by a gap within which the well 120 is positioned. The pair of electrodes are configured to apply an electrical stimulation to tissues within the one or more wells of the device 106 (e.g., the tissue 130 within the well 120 shown in the expanded portion 106-1). During maturation of the tissues within the device 106, the pair of electrodes apply stimulation to the tissues according to a multi-week electrical stimulation protocol. After the tissues are matured, the pair of electrodes may be configured to stimulate the tissues (e.g., the tissue 130) at a set frequency, or pacing frequency. In one embodiment, the frequency, or pacing frequency, at which the tissues are stimulated is set by the control unit 104. As such, the control unit 104 may be configured to send an instruction 132 to the bioreactor 102 to cause the bioreactor 102 to stimulate the tissue, or tissues, within the device 106 at a set pacing frequency.

The first element 126-1 and the second element 126-2 are disposed across the well 120 such that there is a gap between the bottom of the well 120 and the pair of elements. The first element 126-1 and the second element 126-2 are configured to: (a) permit attachment of the tissue 130 formed therebetween, thereby suspending the tissue 130 above the bottom of the well 120, and (b) deform in response to the contractile force exerted on the pair of elements by the tissue 130, thereby simulating a physiological environment that is native to the tissue 130 and/or permitting measurement of the contractile force (e.g., by the sensor assembly 108). For example, the pair of electrodes may subject the tissue 130 to an electrical stimulation at a frequency of 0.1 Hz. The tissue 130 will contract in response to this electrical stimulation causing deformation of at least one of the first element 126-1 and the second element 126-2. Measuring the deformation of the pair of elements allows the functional response of the tissue 130 when stimulated at 0.1 Hz to be recorded.

The sensor assembly 108 is configured to detect one or more functional responses of a tissue within the device 106 (e.g., one or more functional responses of the tissue 130). In one embodiment, the sensor assembly 108 comprises an optical sensor. The optical sensor is configured to detect a deformation of the first element 126-1 and/or the second element 126-2 (e.g., occurring as a result of contractile force exerted on the first element 126-1 and/or the second element 126-2 by the tissue 130). Detecting the deformation of the first element 126-1 and/or the second element 126-2 allows one or more functional responses such as a displacement, or contractile displacement, of the tissue 130 or a contractile force of the tissue 130 to be determined. Additionally, or alternatively, the optical sensor of the sensor assembly 108 is configured to detect a fluorescence intensity of the tissue 130. Detecting the fluorescence intensity of the tissue 130 allows one or more functional responses such as a transient calcium response of the tissue 130 or a change in membrane potential of the tissue 130 to be determined. Additionally, or alternatively, the optical sensor of the sensor assembly 108 is configured to detect a change in dimensions of the tissue 130 over a time frame. Detecting the change in dimensions of the tissue 130 over the time frame allows one or more functional responses such as the displacement, or contractile displacement, of the tissue 130 or the contractile force of the tissue 130 to be determined.

The optical sensor of the sensor assembly 108 is configured to obtain a plurality of image-based-representations of the one or more functional responses of a tissue (e.g., the tissue 130) over a time frame or time period. For example, the optical sensor may be configured to capture an image, or frame, of a tissue every n seconds. Here, n is associated with a predetermined rate at which the images or frames are to be captured. For example, when n=1 then one image of the tissue is captured per second. Any suitable value of n may be chosen such as n={ 1/60, 1/50, 1/30, 1/24, 1/12, ¼, ½, 1, 2} and the like. In one embodiment, the frame rate is determined according to the frequency at which the tissues within the device are being stimulated. The sequences of images or frames of a tissue over a time frame therefore captures one or more functional responses of the tissue over the time frame.

In one embodiment, the bioreactor 102 is configured to transform the sequence of images which capture the one or more functional responses of the tissue over the time frame to a waveform representation. For example, the sensor assembly 108, the interface 110, or another component of the bioreactor 102 may process an image within the sequence of images to extract features relating to the functional response of the tissue at a time point associated with the image. Features relating to the functional response of the tissue extracted from the sequence of images may then be combined to form a waveform comprising the one or functional responses of the tissue over time. The waveform, such as the waveform 134, may then be output from the bioreactor 102. In an alternative embodiment, the raw image or frame data is output from the bioreactor 102 and a separate unit—e.g., the control unit 104 or an image analysis unit (not shown)—processes this data to determine the time-series functional response data. In this way, the waveforms are transformed or reduced to a different state or thing, e.g., the generation or creation of corresponding spectral representation(s) that provide frequency-based characterizations of the functional responses of the tissue to encode the behavior of the tissue.

The time-series functional response data produced by the bioreactor 102 (e.g., the waveform 134) provides a high fidelity and high information density representation of a functional response of an engineered tissue over a predetermined time period (e.g., the contractile force produced by a first tissue in response to a pacing frequency of 1 Hz over a 30 second period). To obtain such waveform data from the bioreactor 102 for the tissue 130, a command may be sent to the bioreactor 102 (e.g., from the control unit 104) to begin stimulating the tissue 130 at a pacing frequency. Alternatively, no electrical stimulation is applied so as to observe the spontaneous response of the tissue 130. The sensor assembly 108 then captures a sequence of images of the tissue 130 over the predetermined time period. Decisively, the sequence of images captures the response (e.g., deformation) of the first element 126-1 and/or the second element 126-2 as a result of the contractile response of the tissue 130. The sequence of images are then processed to extract the response of the first element 126-1 and/or the second element 126-2 over the predetermined time period and convert the elements' responses over time to a time-series of functional response over time. For example, the displacement of the first element 1261 and/or the second element 126-2 may be used to determine the force of the contractile response of the tissue 130. The time-series (e.g., the waveform 134) is then output from the bioreactor 102 for further processing and/or analysis.

A tissue within the bioreactor 102 (e.g., the tissue 130) may be periodically dosed with a drug or compound and the functional response of the tissue after dosing recorded in the form of a waveform. Alternatively, the functional response of the tissue in the absence of any external dosing regimen may be periodically recorded. In either case, the changes in functional response of the tissue are captured in subsequent waveforms, as illustrated in FIG. 1B.

FIG. 1B shows an example protocol carried out by the system 100 according to embodiments of the present disclosure.

The example protocol shown in FIG. 1B is carried out in relation to a single engineered tissue grown within a bioreactor (e.g., the bioreactor 102 of FIG. 1A). Over a period of n time points, the functional responses of an engineered tissue within the bioreactor are recorded as one or more waveforms. For example, a waveform corresponding to the contractile force of the engineered tissue in response to a 1 Hz electrical stimulation over 30 seconds. At each time point, the engineered tissue may be dosed by a compound or drug prior to recordal of the functional response. As such, the evolution, or change, across the n functional responses represent the change in the response of the tissue in consequence of the dosage regimen.

As shown in FIG. 1B, at time t₁ the engineered tissue is vehicle treated and a first waveform 136-1 is obtained (as described above). A first feature set 138-1 is then obtained from the first waveform 136-1. At time t₂ the engineered tissue is treated with a first dose of a compound, a second waveform 136-2 is obtained, and a second feature set 138-2 is obtained from the second waveform 136-2. At time t₃ the engineered tissue is treated with a second dose of the compound, a third waveform 136-3 is obtained, and a third feature set 138-3 is obtained from the third waveform 136-3. At time tₙ the engineered tissue is treated with a final dose of the compound, a final waveform 136-4 is obtained, and a final feature set 138-4 is obtained from the final waveform 136-4.

The first waveform 136-1, second waveform 136-2, third waveform 136-3, and final waveform 136-4 capture the functional responses of the tissue across the n time points. As will be described in more detail below, analyzing the changes in the engineered tissue's functional response over the different time points allows a signature of the tissue's behavior in response to the compound to be captured.

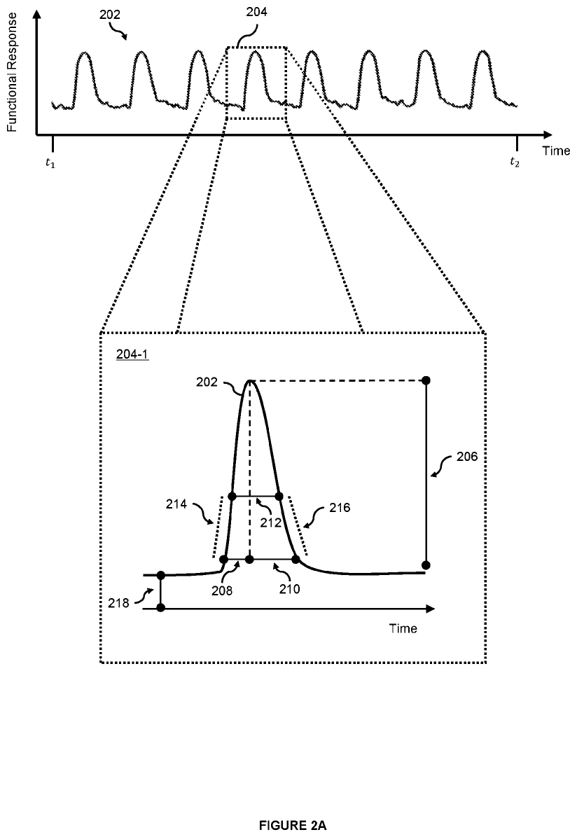

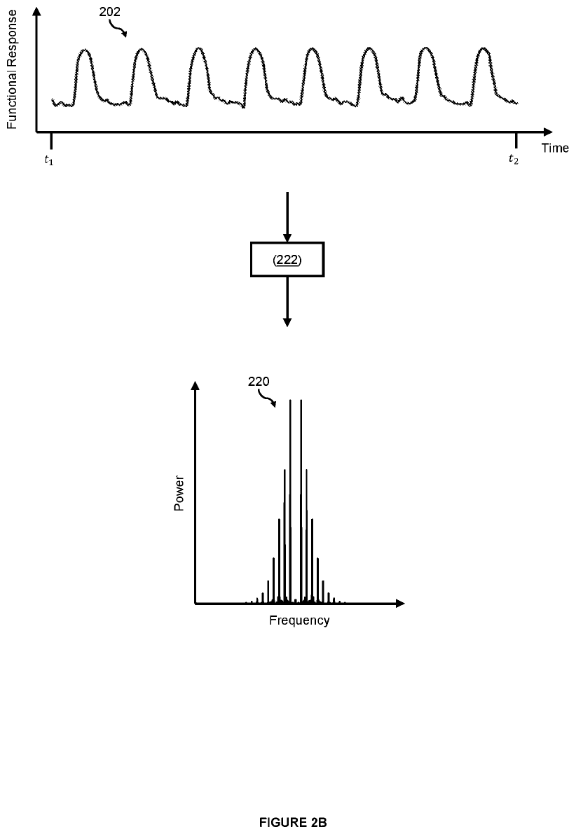

FIG. 2A shows a waveform 202 comprising a functional response of a tissue over a predetermined time period (e.g., the first waveform 136-1 shown in FIG. 1B).

The waveform 202 is obtained from a bioreactor such as the bioreactor 102 shown in FIG. 1A as described above. The waveform 202 provides a high fidelity and high information density representation of the functional response of the tissue over the time frame t₁ to t₂. The waveform 202 comprises a plurality of peaks corresponding to the contractile response of the tissue over the time frame (e.g., 30 s, 40 s, etc.). A magnification of a peak within the portion 204 of the waveform 202 is shown in the expanded portion 204-1.

Typically, a plurality of features is extracted from each peak of the waveform 202 to characterize the waveform 202 and thereby allow for further processing or analysis of the waveform 202. In the example described above in relation to FIG. 1B, the plurality of features extracted for the first waveform 136-1 are represented in the first feature set 138-1. As shown in the expanded portion 204-1, the features extracted from a single peak include peak (or twitch) amplitude 206, time to peak amplitude 208 (or contraction time), time to peak decline 210 (or relaxation time), duration 212 (or twitch duration), maximum rate of development 214 (or maximum contraction slope), maximum rate of declination 216 (or maximum relaxation slope), and passive tension 218.

Extraction of these features from a waveform requires an accurate identification of specific key points on the waveform (e.g., accurately identifying the maximum of a peak, the half peak points, etc.). However, the ability to identify these key points is affected by the noise level in the waveform. Moreover, further analysis is required to capture and represent complex tissue behavior, such as ectopic beats in cardiomyocyte waveforms, from these features. As such, representing the noisy and complex periodic behavior of a tissue's functional response using such feature extraction approaches may not provide an accurate characterization of the behavior of the tissue. Consequently, certain features may be missed when using standard feature extraction approaches, or complex behaviors may be ignored in subsequent processing. This may in turn lead to inaccurate reporting of the behavior of the tissue. As such, these feature extraction approaches may not be suitable for use in protocols such as that described in relation to FIG. 1B.

Therefore, the present disclosure describes a robust feature representation which provides an efficient and high fidelity representation of a tissue's functional response. The feature representation is in the form of a spectral representation, or spectral fingerprint, determined from a waveform of a functional response of a tissue over a time frame.

FIG. 2B shows a spectral representation 220 of the waveform 202 shown in FIG. 2A according to embodiments of the present disclosure.

The spectral representation 220 of the waveform 202 shown in FIG. 2A is obtained from a spectral transformation 222 of the waveform 202. As shown in FIG. 2B, the spectral representation 220 provides a frequency-based characterization of the functional response of the tissue during the time frame t₁ to t₂.

The spectral representation 220 provides a compact, high fidelity, and accurate encoding of a tissue's behavior (e.g., the periodicity of the tissue's contractile response, abnormalities in the periodicity, etc.). Encoding may comprise a numerical representation (vectorization) of data (e.g., stored in memory 1106) that represents or defines tissue response over time, and that may be performed for one or more different conditions, types of tissue response, and/or measurement systems, for example, as shown herein. Furthermore, a spectral representation of a tissue's functional response encodes complex behavior which may not be identified using the above mentioned standard time-series feature extraction processes. In the example shown in FIG. 1B, using spectral representations in place of the feature sets (e.g., the first feature set 138-1, the second feature set 138-2, etc.) allows the functional responses of the tissue at different time points to be effectively and efficiently compared thereby allowing a signature, or representation, of the change in behavior of the tissue as a result of the dosage regimen to be captured. This allows spectral representations to be effectively utilized in a number of downstream tasks and processes, such as drug development and drug discovery.

Referring once again to FIG. 1A, the transformation module 112 is configured to obtain a spectral representation of a waveform (e.g., the waveform 134) by applying a spectral transformation to the waveform. In one embodiment, the spectral transformation comprises a Fourier transform applied to the waveform. In an alternative embodiment, the spectral transformation comprises a maximum entropy transform applied to the waveform.

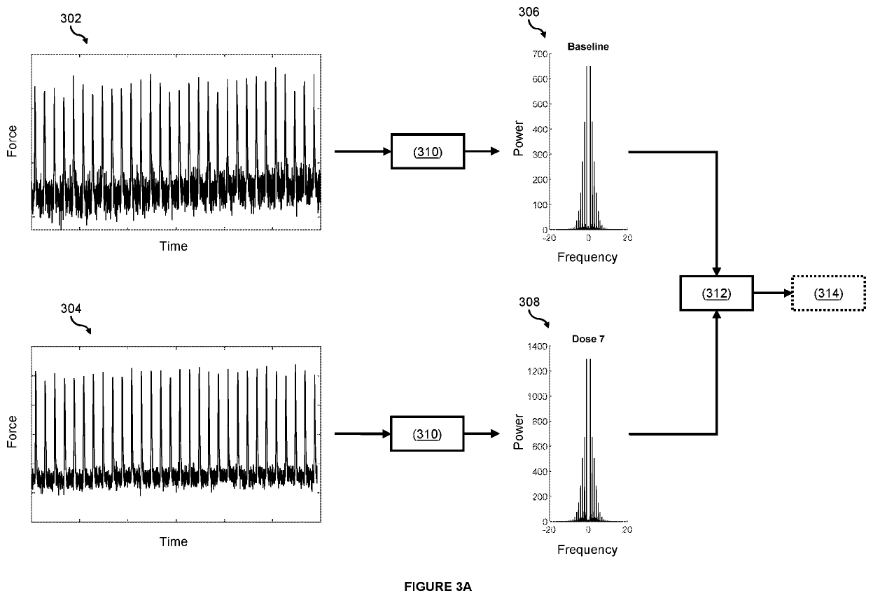

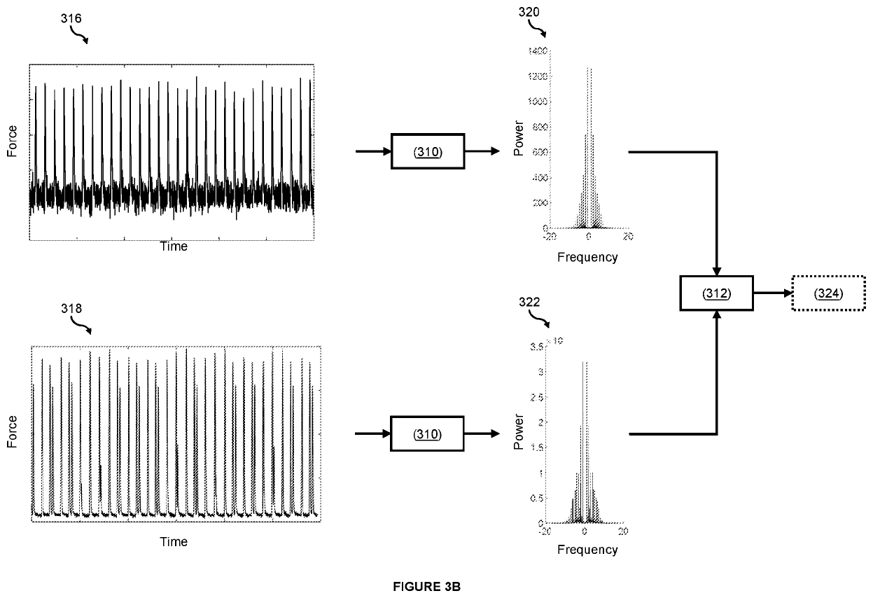

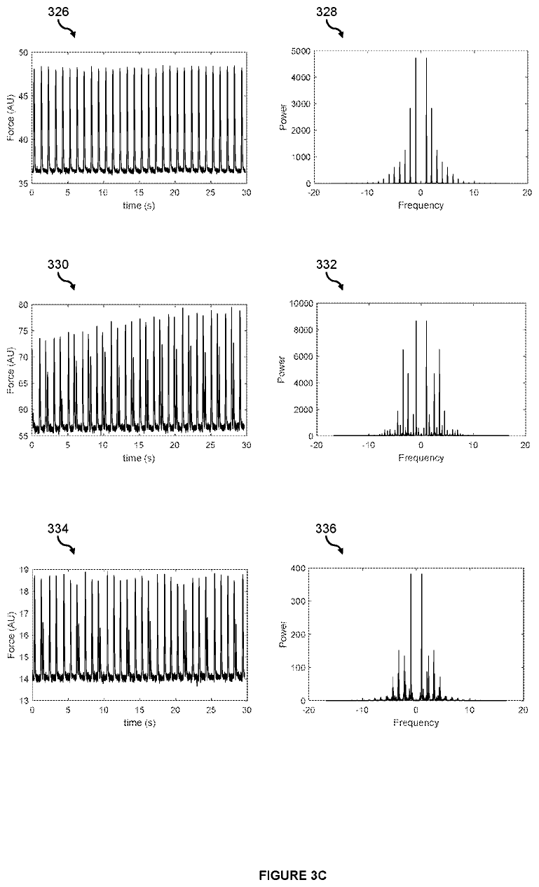

The control unit 104 then outputs the spectral representation to another module, such as the shift detection module 114, the grouping module 116 or the prediction module 118. The functionality of each of these modules is described in more detail in relation to FIGS. 3-5 below. The control unit 104 may then output the one or more spectral representations for use in a drug discovery or development process (as indicated by the dashed lines extending from the control unit 104 in FIG. 1A).

FIG. 3A illustrates a process for determining a relative spectral representation, or signature, of a tissue according to embodiments of the present disclosure.

FIG. 3A shows a first waveform 302 associated with a functional response of a first engineered tissue at a first time point, a second waveform 304 associated with the functional response of the first engineered tissue at a second time point, a first spectral representation 306, and a second spectral representation 308. FIG. 3A further shows a transformation process 310 (e.g., a transformation process performed by the transformation module 112 of the control unit 104 in FIG. 1A), a shift detection process 312 (e.g., the shift detection module 114 of the control unit 104 in FIG. 1A), and a third spectral representation 314 determined by the shift detection process 312. The first spectral representation 306 corresponds to a spectral transformation of the first waveform 302 found using the transformation process 310. The second spectral representation 308 corresponds to a spectral transformation of the second waveform 304 found using the transformation process 310. The first spectral representation 306 and the second spectral representation 308 are then analyzed by the shift detection process 312 to determine the third spectral representation 314.

In general, the process illustrated in FIG. 3A determines a relative spectral representation, or signature, for the first engineered tissue. The relative spectral representation encodes the change in functional response of the first engineered tissue between a first time point and a second time point by comparing spectral representations determined from functional response waveforms obtained from the engineered tissue at each time point. For example, a spectral representation obtained at a time point tₙ (e.g., a spectral representation obtained from the final waveform 136-4 shown in FIG. 1B) may be compared against a spectral representation obtained at time point t₁ (e.g., a spectral representation obtained from the first waveform 136-4 shown in FIG. 1B) to determine a change in behavior of the first engineered tissue between the two time points. This change in behavior may then be linked to an intervention applied to the first engineered tissue between the two time points. Moreover, comparing changes relative to a baseline functional response (e.g., relative to the vehicle treated tissue at time point t₁) allows a relative and comparable signature, or representation, of the tissue's change in behavior to be determined.

In the example of FIG. 3A, the first waveform 302 corresponds to the contractile response (force) of the first engineered tissue over a first time period of 30 seconds. The first engineered tissue is an engineered cardiomyocyte tissue stimulated at a pacing frequency of 1 Hz. As such, the peaks within the first waveform correspond to the “beats” of the engineered cardiomyocyte tissue occurring in response to the 1 Hz electrical stimulation. The first engineered tissue was vehicle treated prior to recording the first waveform 302. As such, the first waveform 302 may be understood as a control or baseline response of the first engineered tissue obtained at a first time point. The first spectral representation 306 of the first waveform 302 therefore provides an encoding of this baseline response. As can be seen from the first spectral representation 306, the power is strongest at 1 Hz (i.e., the pacing frequency) and diminishes with increasing harmonics. The baseline response encoded in the first spectral representation 306 provides an important characterization of the tissue's behavior in the absence of any intervention. As will be describe in more detail below, this baseline response is used to provide a control against which subsequent behaviors of the tissue is compared.

The second waveform 304 corresponds to the contractile response (force) of the first engineered tissue over a second time period of 30 seconds. The second waveform 304 was obtained at a second time point occurring after the first time point. As with the first waveform 302, the first engineered tissue was stimulated at a 1 Hz pacing frequency during the recordal of the second waveform 304. In the example shown in FIG. 3A, no intervention (e.g., drug) was applied to the first engineered tissue between the first and the second time period. As with the first spectral representation 306, the power in the second spectral representation 308 is strongest at 1 Hz and diminishes with increasing harmonics.

The first spectral representation 306 and the second spectral representation 308 are then compared at the shift detection process 312 to determine the third spectral representation 314. The operations performed by the shift detection process 312 identify relative shifts, or changes, in frequency responses in the second spectral representation 308 as compared to the first spectral representation 306 (i.e., the baseline representation). Since the first spectral representation 306 and the second spectral representation 308 are obtained with respect to the same tissue, the shifts in the spectral representations may then be linked to a potential change in behavior in the tissue. In one embodiment, the shift detection process 312 determines the third spectral representation 314 by identifying the relative change—i.e., the relative difference—between the second spectral representation 308 and the first spectral representation 306. For example, the third spectral representation 314 may be the difference between the two spectral representations identified by subtracting the second spectral representation 308 from the first spectral representation 306. Alternatively, the third spectral representation 314 may correspond to the relative change between the two spectral representations determined by dividing the difference between the second spectral representation 308 and the first spectral representation 306 by the first spectral representation 306.

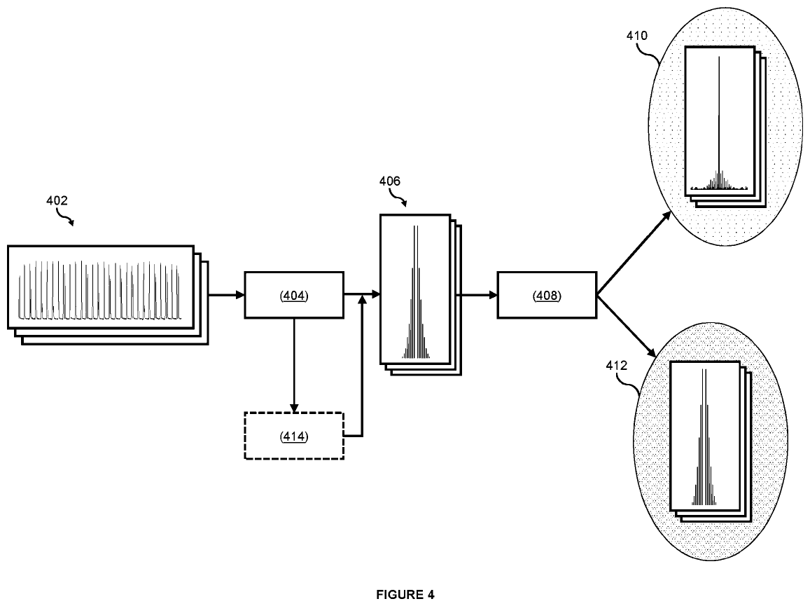

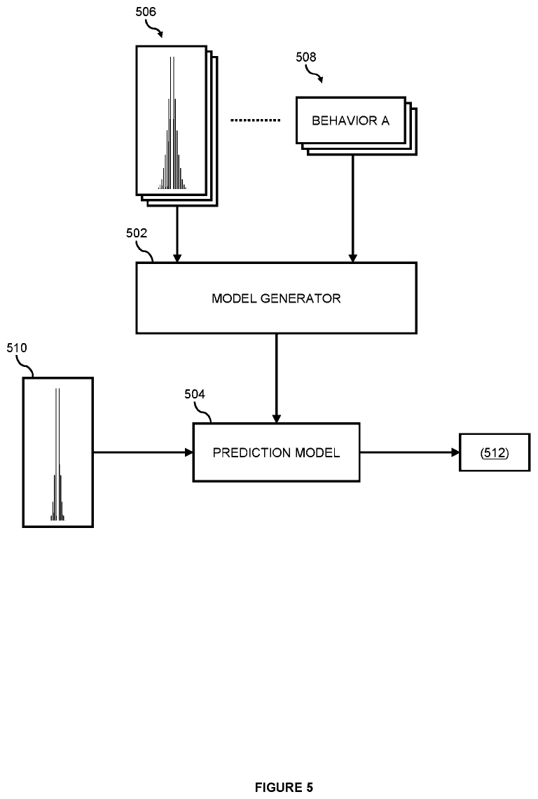

Identifying shifts occurring in the second spectral representation 308 as compared to the first spectral representation 306 helps identify changes in behavior of the first engineered tissue between the times at which the first waveform 302 and the second waveform 304 were obtained. For example, a shift in the third spectral representation 314 away from the primary harmonic (i.e., the stimulating, or pacing, frequency) to other harmonics may indicate longer duration waveforms, whilst a general shift away from the harmonics may indicate less forceful contractions. As a further example, strong peaks outside the harmonics within the third spectral representation 314 may indicate arrhythmias (i.e., contractions occurring outside the stimulating frequency. The third spectral representation 314 (alternatively referred to as a signature, a relative representation, or an output representation) therefore encodes the change in behavior of the first engineered tissue between the first time point and the second time point. This signature provides a relative characterization of the change in behavior which can be used for tasks such as grouping, classifying, and predicting tissue behaviors (as described below in relation to FIGS. 4 and 5).

In the example shown in FIG. 3A, because the second spectral representation 308 is similar to the first spectral representation 306, the change in behavior encoded in the third spectral representation 314 is minimal. It can thus be inferred that no significant change in behavior of the first engineered tissue has occurred between the two time points.

FIG. 3B illustrates a further example of a relative spectral representation according to embodiments of the present disclosure.

FIG. 3B shows a first waveform 316 associated with a functional response of a second engineered tissue at a first time point, a second waveform 318 associated with the functional response of the second engineered tissue at a second time point, a first spectral representation 320, a second spectral representation 322, and a third spectral representation 324. FIG. 3B also shows the transformation process 310 (e.g., the transformation module 112 of the control unit 104 in FIG. 1A) and the shift detection process 312 (e.g., the shift detection module 114 of the control unit 104 in FIG. 1A) shown in FIG. 3A. The first spectral representation 320 corresponds to a spectral transformation of the first waveform 316 found using the transformation process 310 (e.g., the transformation module 112 of the control unit 104 in FIG. 1A). The second spectral representation 322 corresponds to a spectral transformation of the second waveform 318 found using the transformation process 310. The first spectral representation 320 and the second spectral representation 322 are analyzed by the shift detection process 312 to determine the third spectral representation 324.

In the example shown in FIG. 3B, the first waveform 316 corresponds to the contractile response (force) of the second engineered tissue over a first time period of 30 seconds. The second engineered tissue is a cardiomyocyte tissue stimulated at a pacing frequency of 1 Hz. The second engineered tissue was vehicle treated prior to recording the first waveform 316. As such, the first waveform 316 corresponds to the control or baseline response of the second engineered tissue. The first spectral representation 320 of the first waveform 316 provides an encoding of this baseline response and may thus be considered a baseline spectral representation for the second engineered tissue. As with the examples in FIG. 3A, the power is strongest at 1 Hz (i.e., the pacing frequency) and diminishes with increasing harmonics.

The second waveform 318 corresponds to the contractile response (force) of the second engineered tissue over a second time period of 30 seconds. As with the first waveform 316, the second engineered tissue was stimulated at a 1 Hz pacing frequency during the recording of the second waveform 318. In the example shown in FIG. 3B, an intervention of isoproterenol was applied to the second engineered tissue between obtaining the first waveform 316 and obtaining the second waveform 318. Specifically, the second waveform 318 corresponds to the functional response of the second after the seventh dose of isoproterenol was applied. As such, the second spectral representation 322 may be considered a treatment spectral representation for the second engineered tissue.

The first spectral representation 320 and the second spectral representation 322 are then analyzed at the shift detection process 312. As with the example in FIG. 3A, the frequency shifts between the first spectral representation 320 and the second spectral representation 322 are determined and represented in the third spectral representation 324.

The third spectral representation 324 encodes the change in behavior between the baseline spectral representation (i.e., the first spectral representation 320) and the treatment spectral representation (i.e., the second spectral representation 322). In contrast to the example in FIG. 3A, any changes identified by the comparison will be attributed to a change in behavior of the second engineered tissue due to the intervention applied to the second tissue (i.e., the dosing of isoproterenol). In the example shown in FIG. 3B, the shifts in spectra encoded in the third spectral representation 324 reveals that isoproterenol induced more stable baseline and increased amplitude (i.e., increased contractile force). However, the shifts also uncover many abnormal and double contractions. This behavior is illustrated in FIG. 3C.

FIG. 3C illustrates the encoding of abnormal tissue behavior in spectral representations according to embodiments of the present disclosure.

FIG. 3C shows a baseline waveform 326, a baseline spectral representation 328 obtained from the baseline waveform 326, a first abnormal waveform 330, a first abnormal spectral representation 332 obtained from the first abnormal waveform 330, a second abnormal waveform 334, and a second abnormal spectral representation 336 obtained from the second abnormal waveform 334. As in the examples in FIGS. 3A and 3B, the tissues from which the different waveforms were obtained were all engineered cardiomyocyte tissues stimulated at the same pacing frequency of 1 Hz.

The baseline waveform 326 and the baseline spectral representation 328 are associated with an engineered tissue which exhibits normal behavior. That is, the functional response of the engineered tissue (i.e., the contractile force) comprises periodic single beats having consistent force. This behavior is encoded in the baseline spectral representation 328 which shows the strongest power at the 1 Hz pacing frequency with power diminishing with increasing harmonics.

In contrast, the first abnormal waveform 330 and the second abnormal waveform 334 are associated with engineered tissues which exhibit abnormal behavior (i.e., abnormal or double beats). Although these are observable within the first abnormal waveform 330 and the second abnormal waveform 334, they are most clearly seen in the first abnormal spectral representation 332 and the second abnormal spectral representation 336. Both abnormal spectral representations do not exhibit the same diminishing power with increasing harmonics as seen in the baseline spectral representation 328. Instead, there is an increase in power at around 2-4 Hz which indicates the presence of abnormal tissue behavior (i.e., double beats). By comparing the baseline spectral representation 328 to the first abnormal spectral representation 332 or the second abnormal spectral representation 336, the change in tissue behavior can be accurately and efficiently identified. Moreover, the comparison of an abnormal spectral representation to a control spectral representation provides a relative signature of the change in tissue behavior. This relative spectral representation, or signature, provides an efficient and comparable feature for grouping and classification of tissue behaviors.

For example, a relative spectral representation of an engineered tissue dosed with a compound having an unknown mechanism of action may be compared to one or more relative spectral representations of one or more engineered tissues dosed with one or more compounds having known mechanisms of action. Due to the high fidelity of the waveforms from which the relative spectral representations are obtained, and the high information density of the relative spectral representations themselves, the similarity between the relative spectral representation and the one or more relative spectral representations may identify a mechanism of action associated with the engineered tissue. Generally, the term “mechanism of action” refers to how a drug (e.g., a dosed compound) or otherwise pharmaceutical effects tissue(s) of the human body, and can refer to the effect at a molecular level.

FIG. 4 shows the grouping of spectral representations according to an aspect of the present disclosure.

FIG. 4 shows a plurality of waveforms 402, a transformation process 404, a plurality of spectral representations 406, a grouping process 408, a first behavioral grouping 410, and a second behavioral grouping 412. FIG. 4 further shows an optional shift detection process 414. The grouping process illustrated in FIG. 4 is associated with the operations performed by the grouping module 116 of the control unit 104 shown in FIG. 1A.

The plurality of waveforms 402 comprise functional responses of a plurality of engineered tissues over time. The transformation process 404 (e.g., the transformation module 112 of the control unit 104 shown in FIG. 1A) transforms the plurality of waveforms 402 to the plurality of spectral representations 406. That is, a spectral transformation process is applied to each waveform within the plurality of waveforms 402 to determine a corresponding spectral transformation of the plurality of spectral representations 406. The grouping process 408 (e.g., the grouping module 116 of the control unit 104 shown in FIG. 1A) analyses the plurality of spectral representations 406 and groups them into at least two behavioral groups including the first behavioral grouping 410 and the second behavioral grouping 412. The groups are determined by the grouping process 408 so as to assign tissues exhibiting similar or the same behavior to the same group. For example, all tissues within the first behavioral grouping 410 may exhibit behavior relating to a loss of evoked contractions whilst all tissues within the second behavioral grouping 412 may exhibit normal behavior. Alternatively, all tissues within the first behavioral grouping 410 may be associated with compounds having a first mechanism of action whilst all tissues within the second behavioral grouping 412 may be associated with compounds having a second, different, mechanism of action.

In one embodiment, each engineered tissue within the plurality of engineered tissues is associated with at least two waveforms within the plurality of waveforms 402—a baseline, or control waveform, obtained from a tissue under control conditions and a treatment waveform obtained from the tissue under treatment conditions (as described above in relation to FIG. 1B). Here, treatment is to be understood in its general sense as being a comparison to control and may or may not correspond to an intervention in relation to the tissue. Therefore, the plurality of spectral representations 406 determined by the transformation process 404 may be input to a shift detection process 414 (e.g., the shift detection process performed by the shift detection module 114 of the control unit 104 in FIG. 1A) where the baseline spectral representation for each tissue is compared to the corresponding treatment spectral representation to determine a relative signature for that tissue (i.e., a relative spectral representation). In such an embodiment, the plurality of spectral representations 406 comprise a plurality of relative signatures, or relative spectral representations for the plurality of engineered tissues.

The grouping process 408 determines the at least two behavioral groups based on the tissue behaviors encoded in the plurality of spectral representations 406. In one embodiment, the grouping process 408 utilizes an unsupervised grouping process to determine the behavioral groupings based on the plurality of spectral representations 406. The grouping process 408 is unsupervised because labelling information (such as behavior) is not available for the plurality of tissues from which the plurality of spectral representations 406 are determined. The grouping process 408 may utilize an unsupervised clustering algorithm, such as k-means clustering, k-medoids clustering, hierarchical clustering, Gaussian mixture models, and the like to determine the groupings. The number of groupings to identify (e.g., 2, 3, 4, etc.) may be provided as a parameter to the clustering algorithm. Alternatively, the number of groupings is learnt from the plurality of spectral representations 406 using an approach such as the elbow method, the gap statistic, cross validation, and the like.

In another embodiment, the grouping process 408 utilizes a semi-supervised grouping process to determine the behavioral groupings based on the plurality of spectral representations 406. The grouping process 408 is semi-supervised because labelling information (such as behavior) is available only for a subset of the plurality of tissues from which the plurality of spectral representations 406 are determined. The grouping process 408 forms the groups by propagating the labelling to other spectral representations based on the similarities. The grouping process 408 may utilize a seeded k-means clustering approach to determine the groupings. Such an approach works by identifying the number of groups (k) from the available labelled information and calculating the centroid for each group (i.e., calculating the mean spectral representation for each group). The k-means clustering algorithm is then run in the normal manner but with the seeded centroids identified for each group acting as the initial estimates of the cluster centers.

In a further embodiment, the grouping process 408 utilizes a supervised grouping process to determine the behavioral groupings. In such a setting, the behavioral grouping information associated with each tissue is directly available such that the at least two behavioral groups can be identified directly using this information.

The behavioral groupings determined by the grouping process 408 may then be used to assign a probable behavior to an engineered tissue having an unknown behavior. For example, the at least behavioral groupings may comprise a first behavioral group associated with engineered tissues treated with compounds having a first mechanism of action and a second behavioral group associated with engineered tissues treated with compounds having a second mechanism of action. A relative spectral representation for an engineered tissue treated using a compound having an unknown mechanism of action is obtained (e.g., as described above in relation to FIGS. 3A and 3B). The similarity of the relative spectral representation to each of the behavioral groups is determined and used to obtain a probability vector for the engineered tissue. The probability vector represents the probability that the compound used to treat the engineered tissue has the first mechanism of action and/or the second mechanism of action. If the probability associated with either of the mechanisms of action exceeds a predetermined threshold, then the compound used to treat the engineered tissue may be associated with the corresponding mechanism of action.

FIG. 5 illustrates the use of a prediction model to assign a probable behavior to an engineered tissue according to an aspect of the present disclosure.

FIG. 5 shows a model generator 502, a prediction model 504, a plurality of spectral representations 506, and a plurality of behaviors 508. FIG. 5 further shows a first spectral representation 510 and a first prediction 512. The prediction process illustrated in FIG. 5 is associated with the operations performed by the prediction module 118 of the control unit 104 shown in FIG. 1A.

Each spectral representation within the plurality of spectral representations 506 is associated with a corresponding behavior within the plurality of behaviors 508. The plurality of behaviors 508 may be considered as labeling information associated with the plurality of spectral representations 506. The model generator 502 is configured to generate the prediction model 504 (e.g., the prediction module 118 of the control unit 104 shown in FIG. 1A) using the plurality of spectral representations 506 and the plurality of behaviors 508. The prediction model 504 is configured to take a spectral representation as input, such as the first spectral representation 510, and provide a prediction as output, such as the first prediction 512. The first prediction 512 comprises a probability that a tissue associated with the first spectral representation 510 is associated with a behavior. The probability may then be used to identify a behavior associated with the tissue. In this way, the systems and methods of the present disclosure improve over conventional technologies at least because prior art systems and methods require an empirical or trial-and-error approach that can involve real-world trials on human tissue that can result in, and require, large database and memory utilization and processor usage to arrive at a similar real-world or simulated results that has a same or similar result. By contrast, the disclosed systems and methods describe growing and testing tissue for defining a limited set of data specific to the tissue (e.g., human engineered tissue), which requires less memory usage and/or processing utilization compared to a conventional approach where large sets of unknown, potentially irrelevant data may be used or required. The prediction model may be deployed on an underlying computing device or system (e.g., computing system 1100), thereby, improving its accuracy and prediction in performing drug discovery and development tasks as described herein.

In one embodiment, the plurality of spectral representations 506 comprise a plurality of direct spectral representations (such as that illustrated by the spectral representation 220 shown in FIG. 2B). In an alternative embodiment, the plurality of spectral representations 506 comprise a plurality of relative spectral representations. That is, each spectral representation within the plurality of spectral representations 506 encodes the change in behavior of a tissue relative to a baseline, or control (as described above in relation to FIGS. 1B, 3A, and 3B).

The plurality of spectral representations 506 and the plurality of behaviors 508 may thus be considered as a training data set of spectral representations with corresponding labelling information. The training data set is composed of spectral representations obtained from several artificial tissues dosed with different drugs or compounds having known behaviors, or mechanisms of action. Different training data sets are utilized for different tissue types (e.g., cardiac tissue, skeletal tissue, etc.) such that only spectral representations for a given tissue type appear within the training data set.

In one embodiment, the prediction model 504 generated by the model generator 502 is a clustering model. As mentioned above in relation to FIG. 4, a clustering model groups the plurality of spectral representations into behavioral groups based on a degree of similarity such that similar or same behaviors are grouped in the same cluster or group. The model generator 502 generates the clustering-based prediction model using a similar approach as described above in relation to FIG. 4. The prediction model 504 then assigns a probability for an input spectral representation (e.g., the first spectral representation 510) based on a distance, similarity, or dissimilarity, between the input spectral representation and each of the groups. In one example involving two behavioral groups, the prediction model 504 assigns the probability based on a first distance between the input spectral representation and a first centroid associated with the first behavior or a second distance between the input spectral representation and a second centroid associated with the second behavior (the first centroid and the second centroid are determined by the clustering model and learnt or otherwise obtained during the model generation process performed by the model generator 502). The input spectral representation may then be assigned a behavior based on which cluster, or group, the prediction model 504 assigns it to.

In another embodiment, the prediction model 504 generated by the model generator is a classifier. In one embodiment, the classifier comprises a machine learning model which is trained by the model generator 502 to learn a mapping from an input space (i.e., the space of spectral representations, or differences between spectral representations) to an output space (i.e., the set of possible behaviors). The machine learning model assigns a probability, or class, for each behavior given an input spectral representation (or spectral representation difference). Examples of suitable machine learning models include k-nearest neighbors, support vector machines (SVMs), random forests, logistic regression, a multilayer perceptron, and the like. As will be appreciated by the skilled person, several training methodologies may be employed to train the machine learning model on the plurality of spectral representations 506 and the corresponding plurality of behaviors 508. For example, a logistic regression model or a multilayer perceptron model may be trained using stochastic gradient descent whilst a random forest model may be trained using bootstrap aggregating (bagging). Once generated, the prediction model 504 (e.g., the prediction module 118 of the control unit 104 shown in FIG. 1A) is then used to assign one or more probabilities that a tissue with an unknown behavior is associated with one or more of the behaviors.

For example, the first spectral representation 510 shown in FIG. 5 may be associated with an engineered cardiomyocyte tissue treated with a novel compound. The prediction model 504 may be used to determine the first prediction 512 which corresponds to a probable behavior associated with the engineered cardiomyocyte tissue. This probable behavior may then be used to determine the response of the engineered cardiomyocyte tissue to the novel compound. For example, the first prediction 512 may indicate that the engineered cardiomyocyte tissue exhibits a behavior associated with a decreased contractile force. Consequently, it can be inferred that the novel compound may result in a decrease in contractile force.

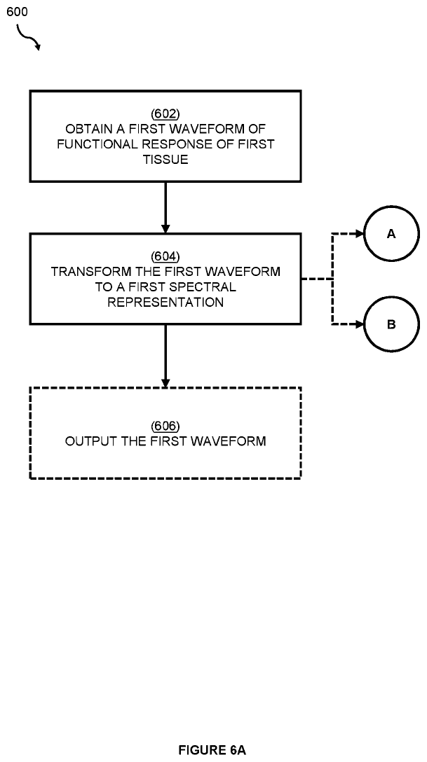

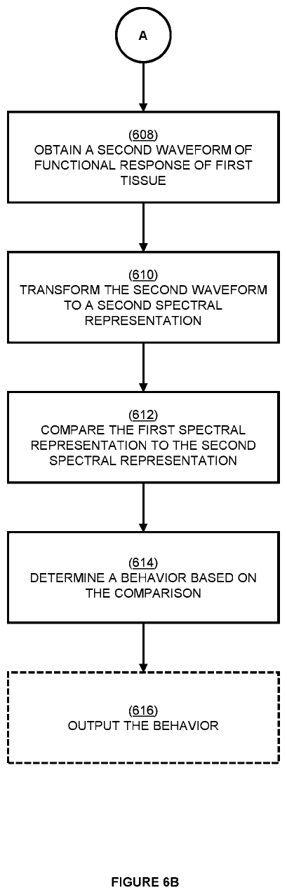

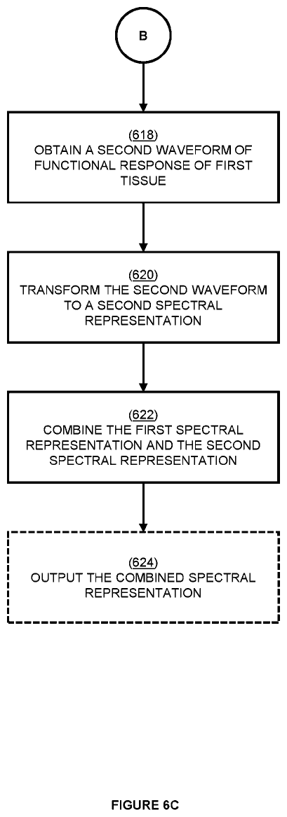

FIG. 6A shows a method 600 for encoding tissue behavior according to an aspect of the present disclosure.

The method 600 comprises the steps of obtaining 602 a first waveform comprising a functional response of a first engineered tissue and transforming 604 the first waveform to a first spectral representation thereby encoding a behavior of the engineered tissue. The method 600 is associated with the examples shown in FIGS. 3A-3B.

The first engineered tissue corresponds to any myopropulsive, or muscle, tissue. The muscle tissue is grown within a device of a bioreactor (e.g., the device 106 of the bioreactor 102 shown in FIG. 1A) from cells seeded therein, such as induced pluripotent stem cells (iPSC).

As such, the first engineered tissue associated with the first waveform corresponds to an engineered tissue. In one embodiment, the engineered tissue is an engineered cardiac tissue.

More particularly, the first engineered tissue may be engineered human cardiac tissue grown from human iPSC-derived cardiomyocytes and ventricular cardiac fibroblasts. Alternatively, the first engineered tissue is engineered skeletal muscle tissue.

At the step of obtaining 602, the first waveform is obtained (e.g., the first waveform 302 shown in FIG. 3A or the first waveform 316 shown in FIG. 3B). The first waveform comprises the functional response of the first engineered tissue over a first time frame (e.g., over 10 seconds, 20 seconds, 30 seconds, 1 minute, etc.).

As stated above in relation to FIG. 1A, the functional response may occur as a result of an electrical stimulation applied to the first engineered tissue at a first pacing frequency (e.g., 1 Hz, 2 Hz, 3 Hz, etc.). Therefore, the method 600 optionally comprises, prior to the step of obtaining 602, the step of stimulating the first tissue at a first pacing frequency such that the first waveform comprises the functional response of the first tissue stimulated at the first pacing frequency. The first pacing frequency is from 0.1 Hz to 20 Hz, and preferably the first pacing frequency is from 1 Hz to 6 Hz. Alternatively, the functional response is not induced by an external stimulation. In the absence of external stimulation, the functional responses capture spontaneous functional responses of the first engineered tissue (e.g., spontaneous beating of an engineered cardiac tissue). In one embodiment, the functional response is a contractile response of the first engineered tissue. In such an embodiment, the contractile response of the first engineered tissue comprises a contractile displacement of the first tissue.

Alternatively, the contractile response of the first engineered tissue comprises a contractile force of the first tissue. In an alternative embodiment, the functional response of the first engineered tissue comprises a transient calcium response of the first tissue. In yet a further embodiment, the functional response of the first engineered tissue comprises a change in membrane potential of the first tissue.

At the step of transforming 604, the first waveform is transformed to a first spectral representation (e.g., the first spectral representation 306 of the first waveform 302 shown in FIG. 3A or the first spectral representation 320 of the first waveform 316 shown in FIG. 3B). The first spectral representation provides a frequency-based characterization of the functional response of the first tissue during the first time frame thereby encoding a behavior of the first tissue.

The step of transforming 604 the first waveform to the first spectral representation comprises obtaining the first spectral representation from a spectral transformation of the first waveform (e.g., the transformation process 310 described in relation to FIGS. 3A and 3B above). That is, a spectral transformation is applied to the first waveform to obtain the first spectral representation. The spectral transformation transforms the waveform from the time domain to the frequency domain. As such, any suitable transformation approach may be used such as a Fourier transform, a maximum entropy transform, and the like.

Optionally, the method 600 further comprises the step of outputting 606 the first spectral representation for use in a drug discovery or development process.

In on embodiment, after the step of transforming 604, the method 600 proceeds to the step shown in FIG. 6B (i.e., route “A” as shown in FIG. 6A). In an alternative embodiment, after the step of transforming 604, the method 600 proceeds to the steps shown in FIG. 6C (i.e., route “B” as shown in FIG. 6A).

FIG. 6B illustrates further steps of method 600 that may be performed according to an embodiment of the present disclosure.

The steps shown in FIG. 6B comprise obtaining 608 a second waveform of a further functional response of the first tissue, transforming 610 the second waveform to a second spectral representation, comparing 612 the first and the second spectral representations, determining 614 a behavior based on the comparison, and optionally outputting 616 the behavior. As such, the steps shown in FIG. 6 correspond to the process of determining a behavioral signature, or relative spectral representation, corresponding to a change in behavior of the first engineered tissue as described above in relation to FIGS. 3A and 3B.

At the step of obtaining 608, a second waveform comprising a functional response of the first engineered tissue over a second time frame is obtained (e.g., the second waveform 304 shown in FIG. 3A or the second waveform 318 shown in FIG. 3B). Preferably, the first tissue was vehicle treated prior to obtaining the first waveform over the first time frame and an intervention, or treatment, is made to the first tissue prior to the second time frame.

At the step of transforming 610, the second waveform is transformed to a second spectral representation (e.g., the second spectral transformation 308 in FIG. 3A or the second spectral transformation 322 in FIG. 3B). The second spectral representation provides a frequency-based characterization of the functional response of the first tissue during the second time frame.

At the step of comparing 612, the first spectral representation is compared to the second spectral representation (e.g., by the shift detection process 314 described in relation to FIGS. 3A and 3B above).

At the step of determining 614, a behavior of the first tissue is determined based on the comparison. As described above, a treatment may be given to the first tissue between the first time period (associated with the first spectral representation) and the second time period (associated with the second spectral representation). As such, the change in behavior of the first tissue identified as a result of the comparison may then be associated with the treatment.

Optionally, the steps further comprise the step of outputting 616 the behavior.

Optionally, the steps shown in FIG. 6 further comprise, prior to the step of obtaining 608, the step of stimulating the first tissue at a second pacing frequency such that the second waveform comprises the functional response of the first tissue stimulated at the second pacing frequency. The second pacing frequency may correspond to the same frequency at which the first tissue was stimulated during the first time period (i.e., the second pacing frequency is the same as the first pacing frequency). As such, both the first and the second waveforms may comprise the functional responses of the first tissue when stimulated at the same frequency over the two different time periods. Alternatively, the first pacing frequency and the second pacing frequency are different. As such, the first and the second waveforms may comprise the functional responses of the first tissue when stimulated at different frequencies over the two different time periods. The skilled person will appreciate that the step of stimulating the first tissue prior to obtaining the first and/or the second waveform is optional, and no stimulation may be applied to the first tissue over the first or the second time periods. In such situations, the waveforms would correspond to the spontaneous functional responses of the first tissue over the first and the second time periods.

FIG. 6C illustrates further steps of method 600 that may be performed according to an embodiment of the present disclosure.

The steps shown in FIG. 6C comprise obtaining 618 a second waveform of a further functional response of the first tissue, transforming 620 the second waveform to a second spectral representation, combining 622 the first and the second spectral representations, and optionally outputting 624 the combined spectral representation.

The steps of obtaining 618 and transforming 620 correspond to the steps of obtaining 608 and transforming 610 described above in relation to FIG. 6.

At the step of combining 618, the first spectral representation and the second spectral representation are combined thereby generating a combined spectral representation of the first tissue. Combining the first spectral representation and the second spectral representation comprises adding, multiplying, or otherwise merging the two spectral representations.

Alternatively, combining the first spectral representation and the second spectral representation comprises determining a difference between the two spectral representations.

Optionally, the steps further comprise the step of outputting 620 the combined spectral representation. For example, the combined spectral representation is output for use in a drug discovery or development process.

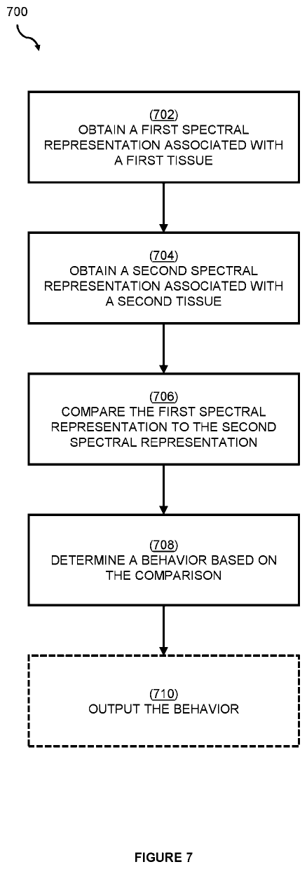

FIG. 7 illustrates a method 700 for determining tissue behavior according to an embodiment of the present disclosure.

The steps shown in FIG. 7 comprise obtaining 702 a first spectral representation associated with a first tissue, obtaining 704 a second spectral representation associated with a second tissue, comparing 706 the first spectral representation and the second spectral representation, determining 708 a behavior based on the comparison, and optionally outputting 710 the determined behavior.

At the step of obtaining 702, the first spectral representation associated with the first tissue is obtained. The first spectral representation is a relative spectral representation as described in relation to FIGS. 3A and 3B above. As such, the first spectral representation is obtained by comparing a treatment spectral representation (i.e., a spectral representation associated with a time point at which a treatment was applied to the first tissue) to a baseline spectral representation (i.e., a spectral representation associated with a prior time point which acts as a control). Alternatively, the first spectral representation is a direct spectral representation.

At the step of obtaining 704, the second spectral representation associated with the second tissue is obtained. The second spectral representation is a relative spectral representation as described above. Alternatively, the first spectral representation is a direct spectral representation. The form of the first spectral representation and the second spectral representation are the same such that, for example, if the first spectral representation is a relative spectral representation then the second spectral representation is also a relative spectral representation.

At the step of comparing 706, the first spectral representation is compared to the second spectral representation. The comparison performed at the step of comparing 706 determines a degree of similarity between the two spectral representations.

At the step of determining 708, a behavior of the first tissue is determined based on the comparison. In one embodiment, the second tissue exhibits the behavior; that is the behavior, which may be associated with an intervention, or treatment, is associated with the second tissue. As such, the behavior of the first tissue is determined to be the behavior associated with the second tissue based on the result of the comparison performed at the step of comparing 706. For example, if the first spectral representation is determined to be similar to the second spectral representation, then it may be inferred that the two tissues from which the spectral representations were obtained exhibit the same behavior.

At the optional step of outputting 710, the behavior of the first tissue determined at the step of determining 708 is output.

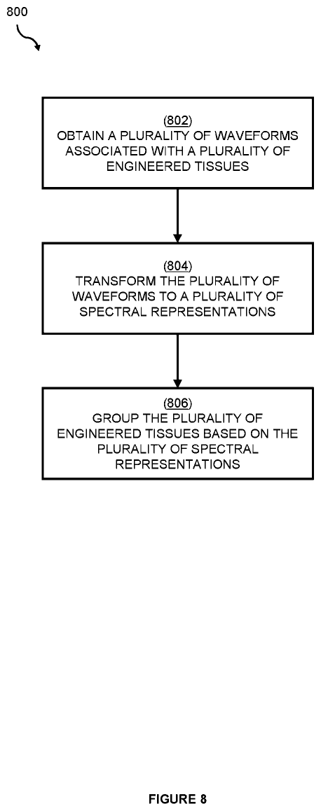

FIG. 8 shows a method 800 for grouping of tissue behaviors using spectral representations according to an aspect of the present disclosure.

The method 800 comprises the steps of obtaining 802 a plurality of associated with a plurality of engineered tissues, transforming 804 the plurality of waveforms to a plurality of spectral representations, and grouping 806 the plurality of engineered tissues based on the plurality of spectral representations.

At the step of obtaining 802, a plurality of waveforms are obtained. The plurality of waveforms comprise functional responses of a plurality of engineered tissues over time. The plurality of waveforms are obtained from one or more bioreactors (e.g., the bioreactor 102 shown in FIG. 1A) and may correspond to the functional responses of the plurality of engineered tissues to a first pacing frequency.

At the step of transforming 804, the plurality of waveforms is transformed to a corresponding plurality of spectral representations.

At the step of grouping 806, the plurality of engineered tissues is grouped into at least two behavioral groups based on the corresponding plurality of spectral representations.

In one embodiment, the at least two behavioral groups comprises a first group associated with a first intervention and a second group associated with a second intervention. For example, the first intervention may correspond to a control, or placebo, group and the second intervention may correspond to a group which received a drug.

Optionally, the method 800 further comprises the step of identifying a behavior associated with a first tissue based on the grouping. The first tissue may be an engineered tissue. The first tissue may be stimulated (e.g., by a bioreactor such as the bioreactor 102 shown in FIG. 1A) at a first pacing frequency, which matches the pacing frequency at which the plurality of engineered tissues were stimulated, such that a waveform obtained in relation to the stimulated first tissue comprises the functional response of the first tissue when stimulated at the first pacing frequency.

As described above in relation to FIG. 4, the step of identifying the behavior associated with the first tissue comprises obtaining a first waveform corresponding to a functional response of the first tissue over a first time frame, transforming the first waveform to a first spectral representation thereof, and identifying the behavior associated with the first tissue based on the grouping and the first spectral representation.

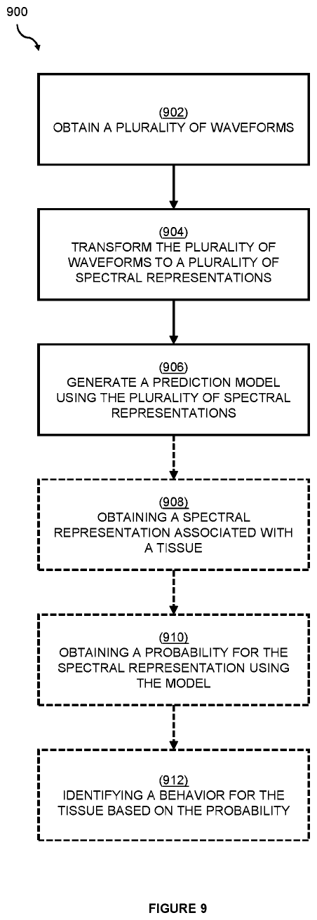

FIG. 9 shows a method 900 for generating a prediction model according to an aspect of the present disclosure.

The method 900 comprises the steps of obtaining 902 a plurality of waveforms, transforming 904 the plurality of waveforms to a plurality of spectral representations, and generating 906 the prediction model using the plurality of spectral representations. Optionally, the method 900 further comprises the steps of obtaining 908 a spectral representation associated with a tissue, obtaining 910 a probability for the tissue using the model, and identifying 912 a behavior for the tissue based on the probability.

At the step of obtaining 902, a plurality of waveforms is obtained. The plurality of waveforms comprise functional responses of a plurality of engineered tissues over time. The plurality of engineered tissues comprise at least a first tissue group associated with a first behavior and a second tissue group associated with a second behavior.

In one embodiment, the first tissue group is associated with a first cell line and the second tissue group is associated with a second cell line. The second cell line is different to the first cell line.

In another embodiment, the first tissue group is associated with a first intervention and the second tissue group is associated with a second intervention.

At the step of transforming 904, the plurality of waveforms is transformed to a corresponding plurality of spectral representations. The corresponding plurality of spectral representations provide frequency-based characterizations of the functional responses of the plurality of engineered tissues.

At the step of generating 906, a prediction model is generated using the corresponding plurality of spectral representations. The prediction model assigns a probability that a tissue exhibits the first behavior, or the second behavior based on a spectral representation of the tissue.

In one embodiment, the prediction model generated comprises a clustering model. The prediction model assigns the probability based on a first distance between the spectral representation of the tissue and a first centroid associated with the first behavior or a second distance between the spectral representation of the tissue and a second centroid associated with the second behavior. The first centroid and the second centroid are determined by the clustering model.

In a further embodiment, the prediction model comprises a classifier. As such, the step of generating the prediction model comprises training the classifier on the corresponding plurality of spectral representations.

At the optional step of obtaining 908, a first spectral representation associated with a first tissue is obtained. The first tissue is associated with an unknown behavior and the first tissue may be an engineered tissue. As stated in detail above, obtaining the first spectral representation may comprise obtaining a first waveform comprising a functional response of the first tissue over a first time frame, and transforming the first waveform to the first spectral representation.

At the optional step of obtaining 910, a first probability associated with the first tissue is obtained from the model based on the first spectral representation.

At the optional step of identifying 912, a behavior associated with the first tissue is identified based on the first probability. The behavior is associated with the first behavior or the second behavior.

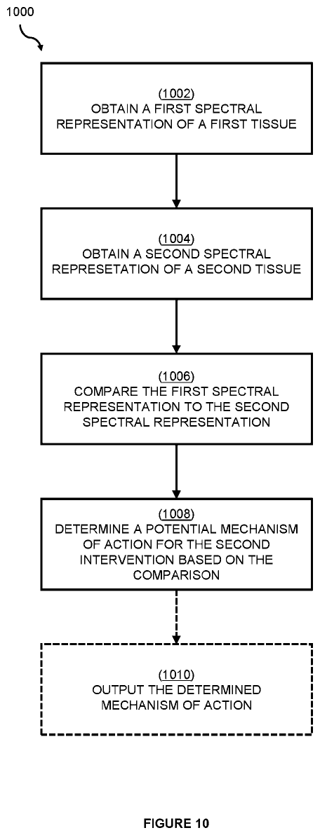

FIG. 10 shows a method 1000 for mechanism of action identification according to an aspect of the present disclosure.

The method 1000 comprising the steps of obtaining 1002 a first spectral representation of a first tissue, obtaining 1004 a second spectral representation of a second tissue, comparing 1006 the first spectral representation to the second spectral representation, and determining 1008 a potential mechanism of action based on the comparison. Optionally, the method 1000 comprises the step of outputting 1010 the potential mechanism of action.

At the step of obtaining 1002, a first spectral representation of a first functional response of a first tissue is obtained. The first tissue is associated with a first intervention having a known mechanism of action. The first tissue is an engineered tissue. The first tissue may be a muscle tissue such as cardiac tissue or skeletal muscle tissue.

The first functional response corresponds to a contractile response of the first tissue such as a contractile force or contractile displacement of the first tissue. Alternatively, the first functional response is a transient calcium response of the first tissue or a change in membrane potential of the first tissue.

As described in detail above, the step of obtaining the first spectral representation may comprise the steps of obtaining a first waveform comprising the first functional response of the first tissue over a first time frame and transforming the first waveform to the first spectral representation. The first spectral representation provides a frequency-based characterization of the first functional response of the first tissue during the first time frame thereby encoding a behavior of the first tissue.

At the step of obtaining 1004, a second spectral representation of a second functional response of a second tissue is obtained. The second tissue is associated with a second intervention having an unknown mechanism of action. The second tissue is an engineered tissue. The second tissue is of the same type as the first tissue and may be a muscle tissue such as cardiac tissue or skeletal muscle tissue.

The second functional response is the same as the first functional response. As such, the second functional response may be a contractile response of the second tissue, such as a contractile force or contractile displacement of the second tissue, a transient calcium response of the second tissue, or a change in membrane potential of the second tissue.

As described in detail above, the step of obtaining the second spectral representation may comprise the steps of obtaining a second waveform comprising the second functional response of the second tissue over a second time frame and transforming the second waveform to the second spectral representation. The second spectral representation provides a frequency-based characterization of the second functional response of the second tissue during the second time frame thereby encoding a behavior of the second tissue.

At the step of comparing 1006, the first spectral representation is compared to the second spectral representation.

At the step of determining 1008, a potential mechanism of action for the second intervention is determined based on the comparing.

At the optional step of outputting 1010, the potential mechanism of action for the second intervention is output.

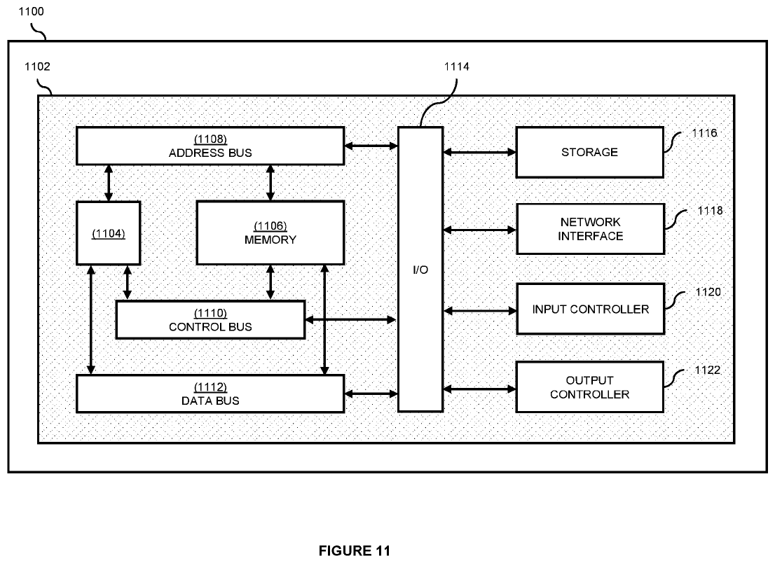

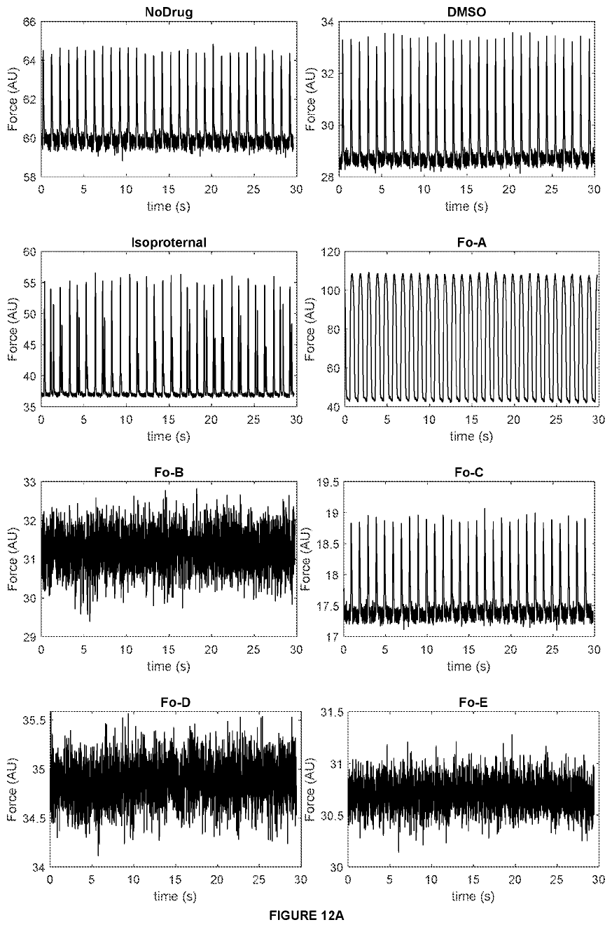

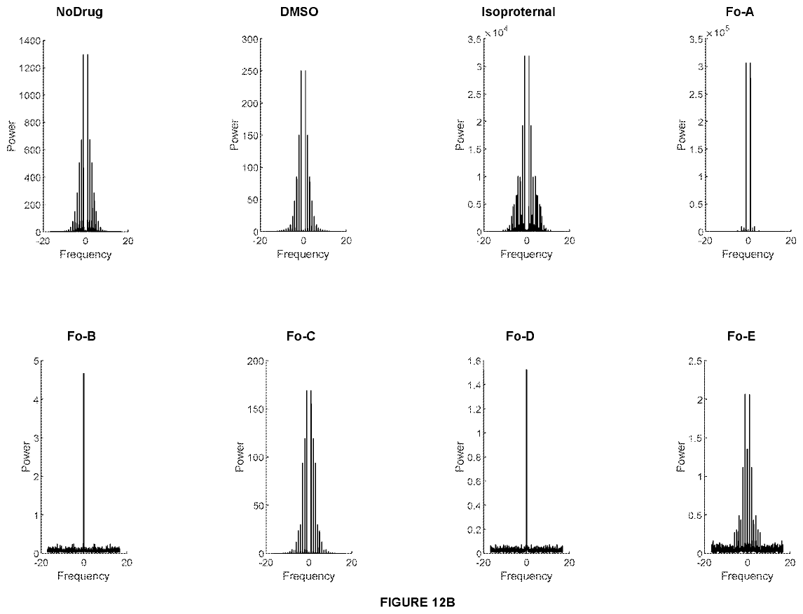

FIG. 11 shows an example computing system for carrying out the methods of the present disclosure. Specifically, FIG. 11 shows a block diagram of an embodiment of a computing system according to example embodiments of the present disclosure.

Computing system 1100 can be configured to perform any of the operations disclosed herein such as, for example, any of the operations discussed with reference to the components described in relation to FIG. 1A. For example, in some aspects computing system 1100 may comprise control unit 104, where, for example, computing system 1100 incorporates the components of control unit 104 and/or any other components of FIG. 1A. Additionally, or alternatively, computing system 1100 may be an external system that is communicatively coupled to control unit 104 and/or any other components of FIG. 1A. Computing system includes one or more computing device(s) 1102. Computing device(s) 1102 of computing system 1100 comprise one or more processors 1104 and memory 1106. One or more processors 1104 can be any general purpose processor(s) configured to execute a set of instructions. For example, one or more processors 1104 can be one or more general-purpose processors, one or more field programmable gate array (FPGA), and/or one or more application specific integrated circuits (ASIC). In one embodiment, one or more processors 1104 include one processor. Alternatively, one or more processors 1104 include a plurality of processors that are operatively connected. One or more processors 1104 are communicatively coupled to memory 1106 via address bus 1108, control bus 1110, and data bus 1112. Memory 1106 can be a random access memory (RAM), a read only memory (ROM), a persistent storage device such as a hard drive, an erasable programmable read only memory (EPROM), and/or the like. Computing device(s) 1102 further comprise I/O interface 1114 communicatively coupled to address bus 1108, control bus 1110, and data bus 1112.

Memory 1106 can store information that can be accessed by one or more processors 1104. For instance, memory 1106 (e.g., one or more non-transitory computer-readable storage mediums, memory devices) can include computer-readable instructions (not shown) that can be executed by one or more processors 1104. The computer-readable instructions can be software written in any suitable programming language or can be implemented in hardware. Additionally, or alternatively, the computer-readable instructions can be executed in logically and/or virtually separate threads on one or more processors 1104. For example, memory 1106 can store instructions (not shown) that when executed by one or more processors 1104 cause one or more processors 1104 to perform operations such as any of the operations and functions for which computing system 1100 is configured, as described herein. In addition, or alternatively, memory 1106 can store data (not shown) that can be obtained, received, accessed, written, manipulated, created, and/or stored. The data can include, for instance, the data and/or information described herein in relation to FIGS. 1 to 14. In some implementations, computing device(s) 1102 can obtain from and/or store data in one or more memory device(s) that are remote from the computing system 1100.

Computing environment 1100 further comprises storage unit 1116, network interface 1118, input controller 1120, and output controller 1122. Storage unit 1116, network interface 1118, input controller 1120, and output controller 1122 are communicatively coupled to central control unit 1102 via I/O interface 1114.

Storage unit 1116 is a computer readable medium, preferably a non-transitory computer readable medium, comprising one or more programs, the one or more programs comprising instructions which when executed by the one or more processors 1104 cause computing environment 1100 to perform the method steps of the present disclosure. Alternatively, storage unit 1116 is a transitory computer readable medium. Storage unit 1116 can be a persistent storage device such as a hard drive, a cloud storage device, or any other appropriate storage device.

Network interface 1118 can be a Wi-Fi module, a network interface card, a Bluetooth module, and/or any other suitable wired or wireless communication device. In an embodiment, network interface 1118 is configured to connect to a network such as a local area network (LAN), or a wide area network (WAN), the Internet, or an intranet.

### EXAMPLE IMPLEMENTATION

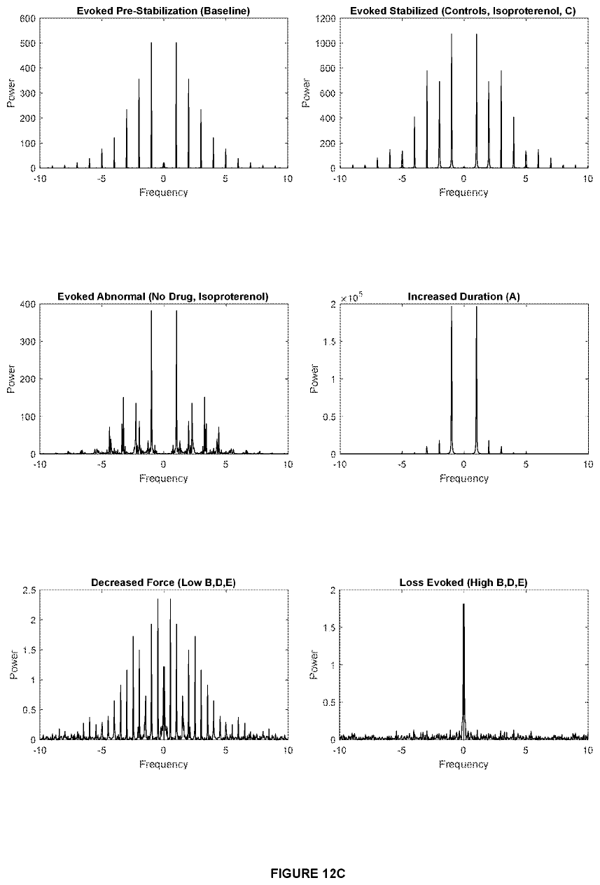

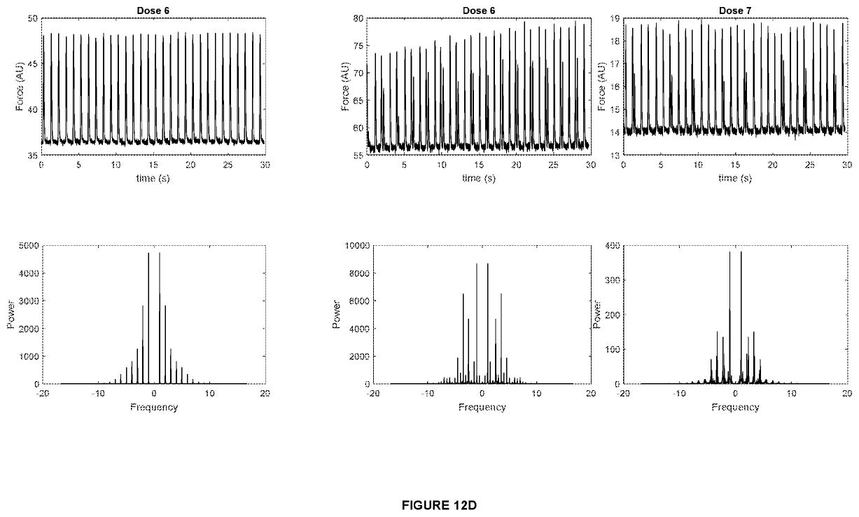

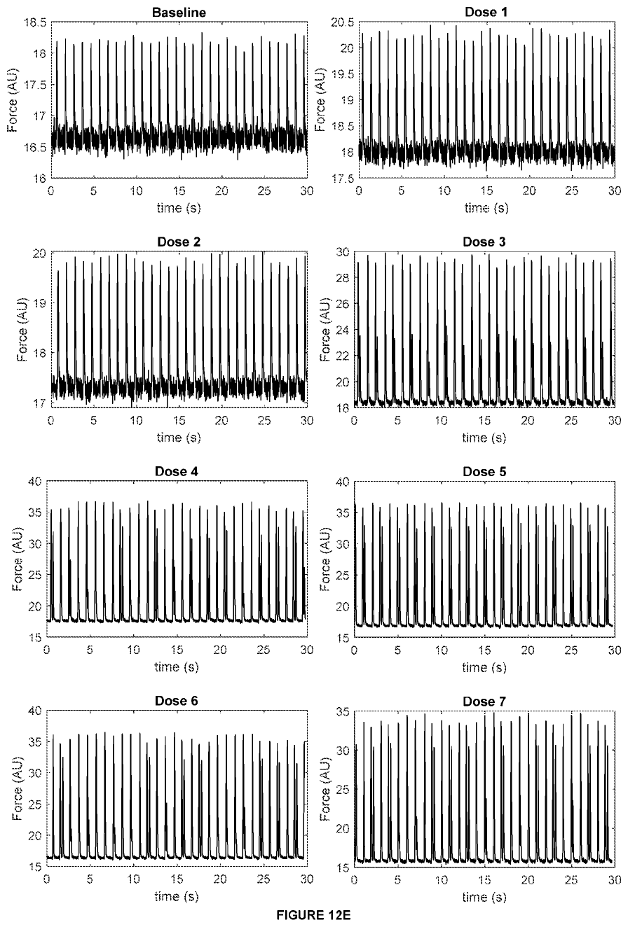

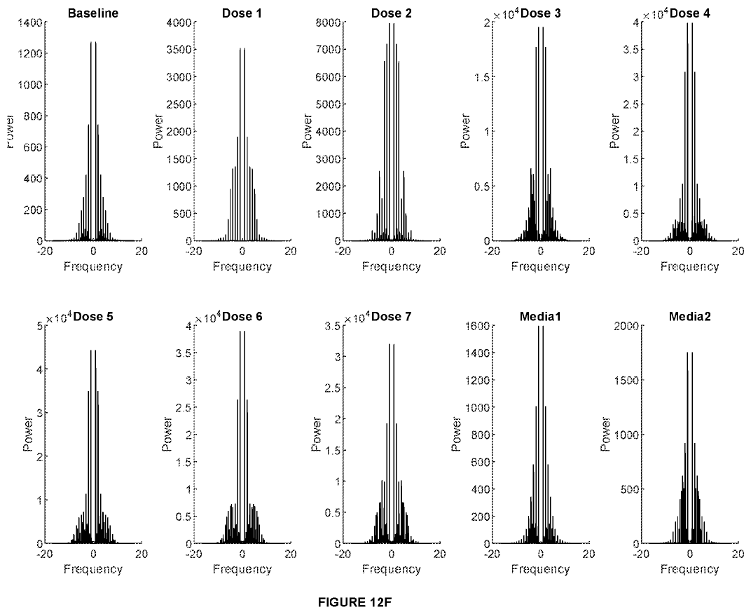

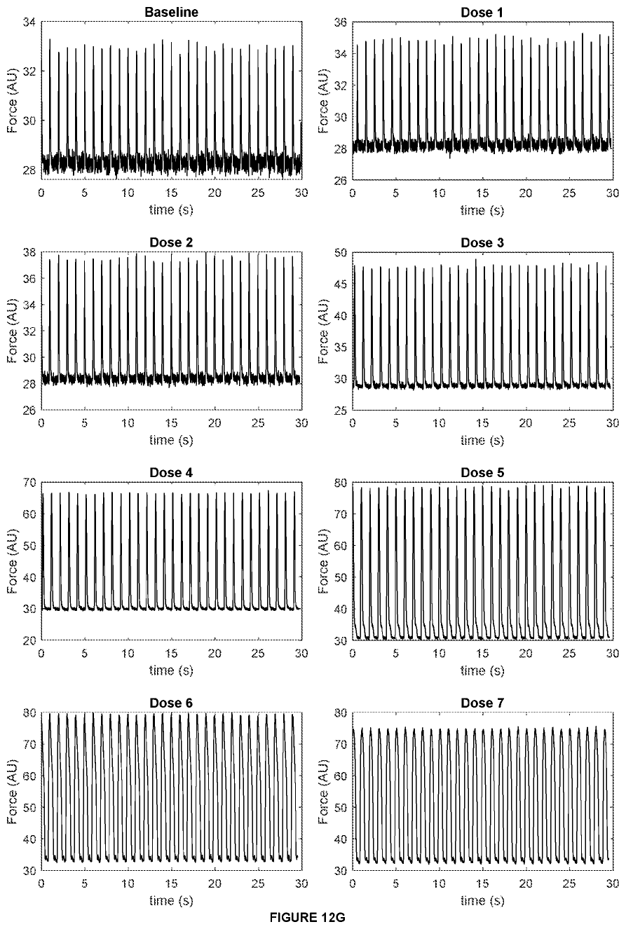

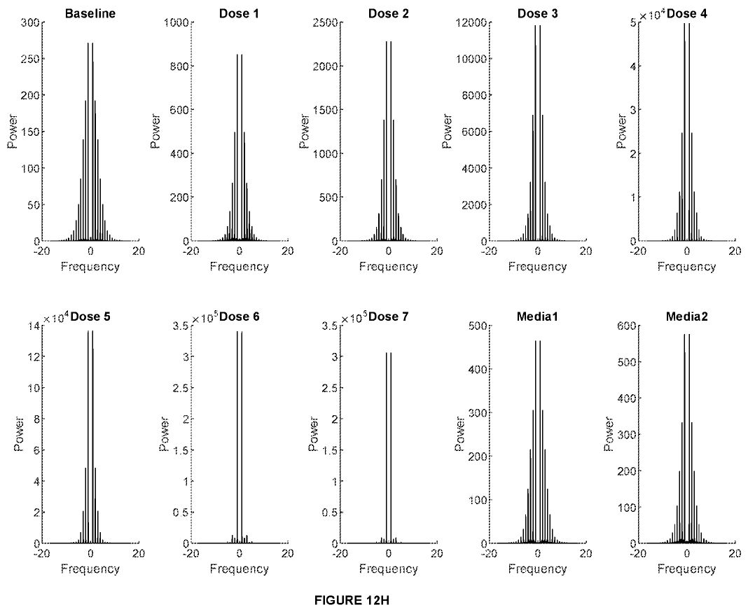

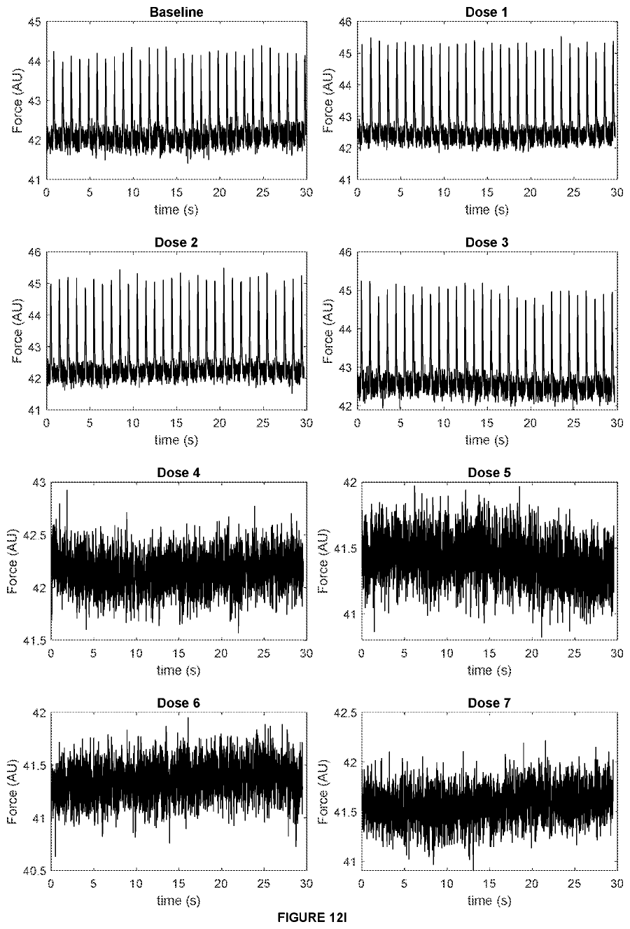

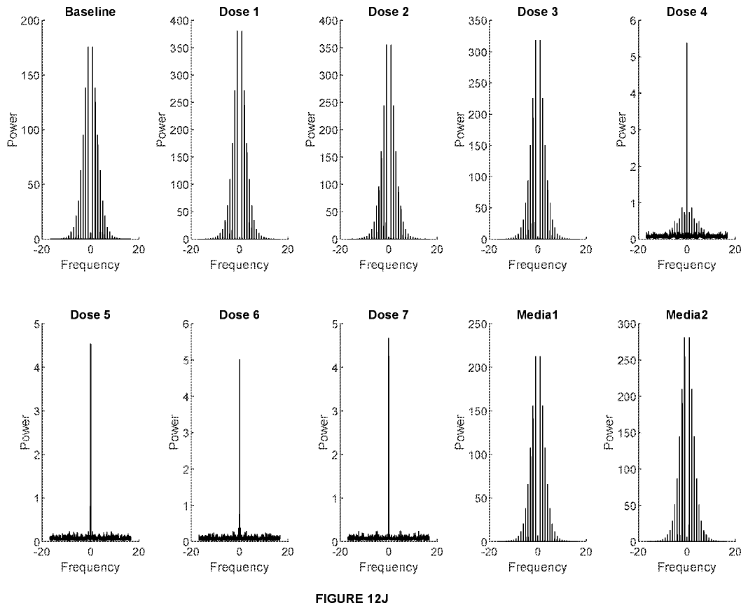

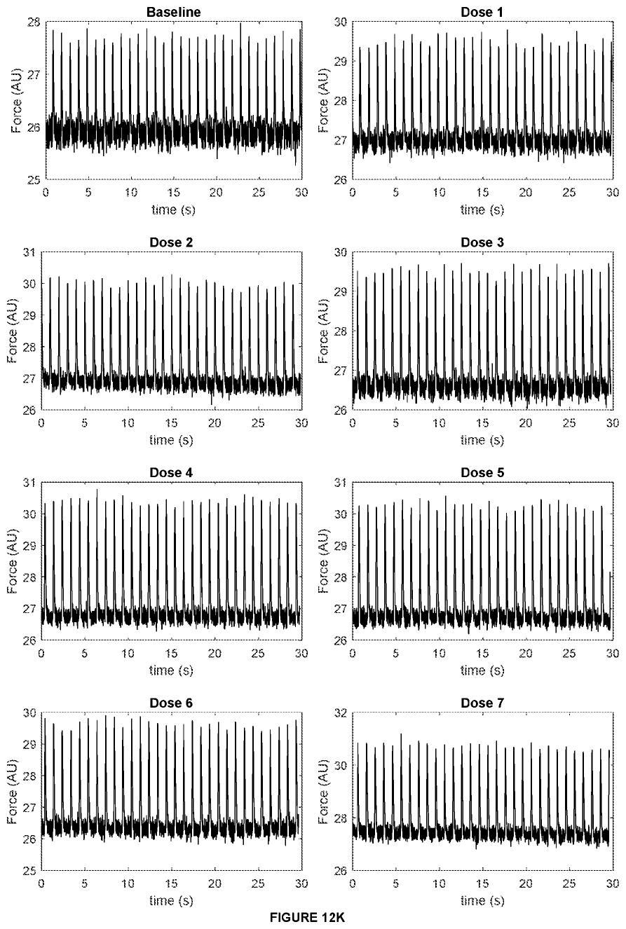

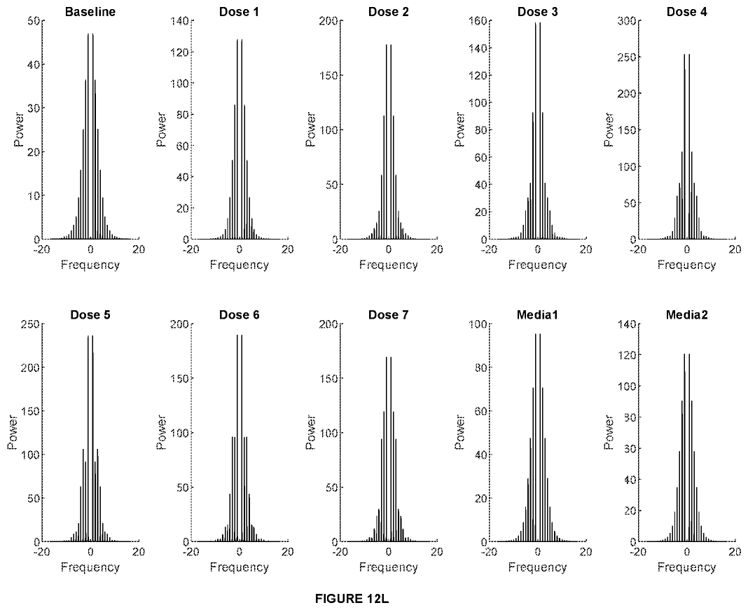

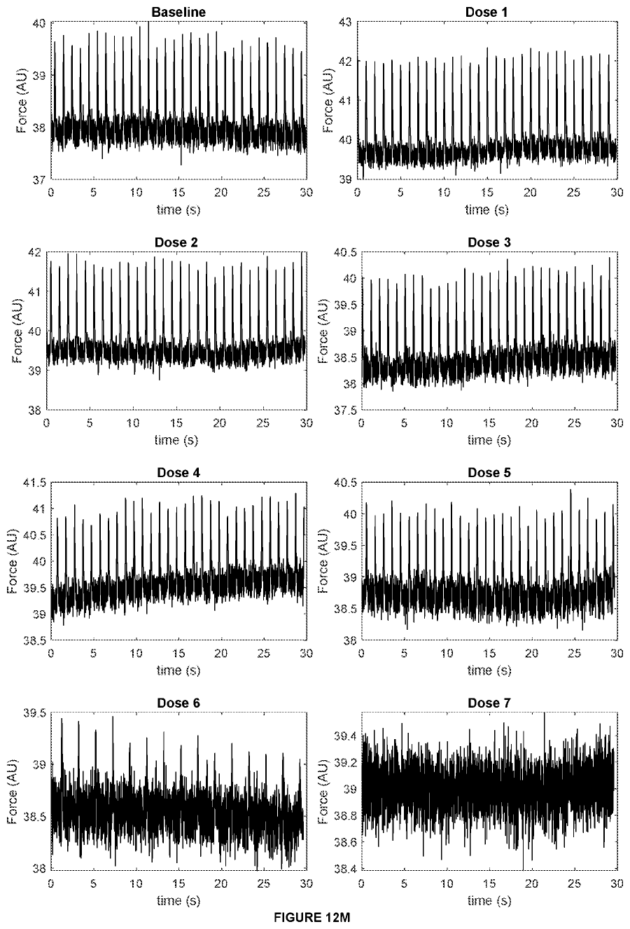

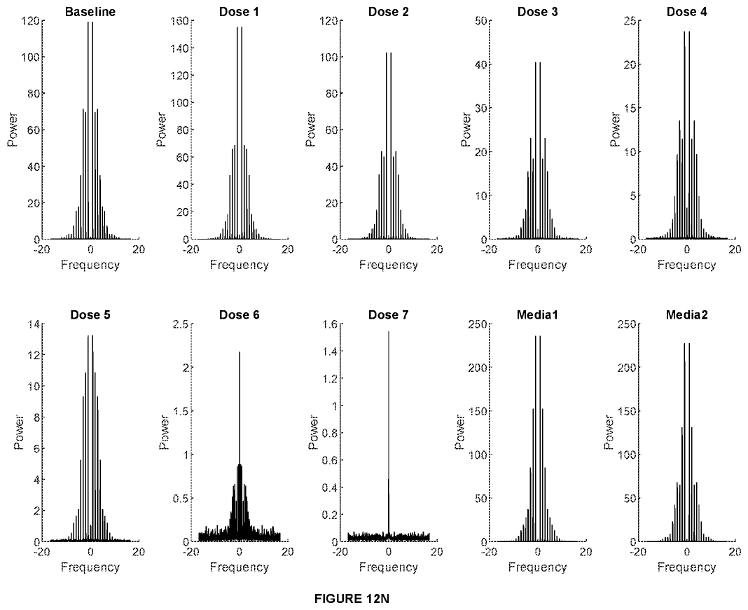

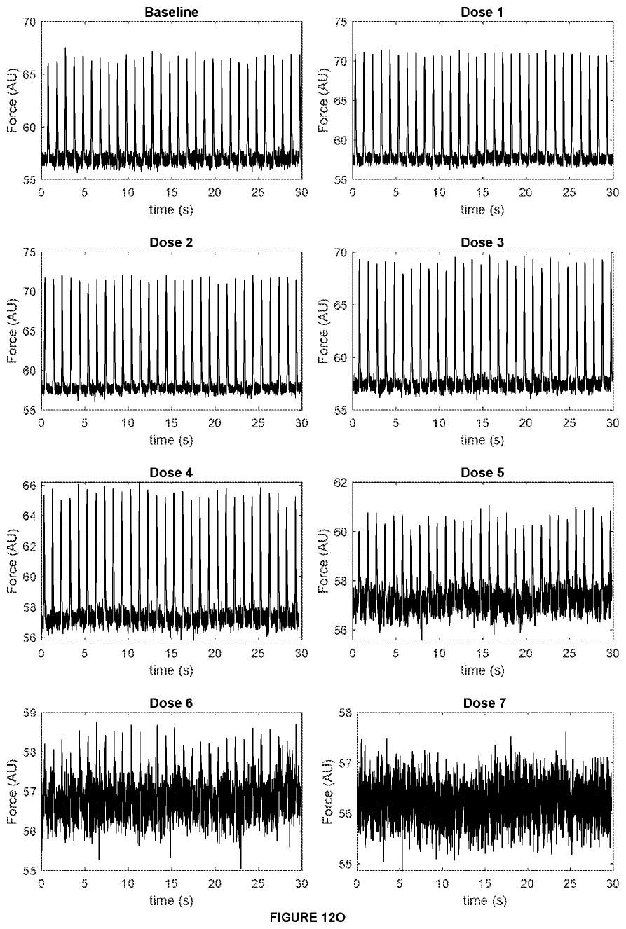

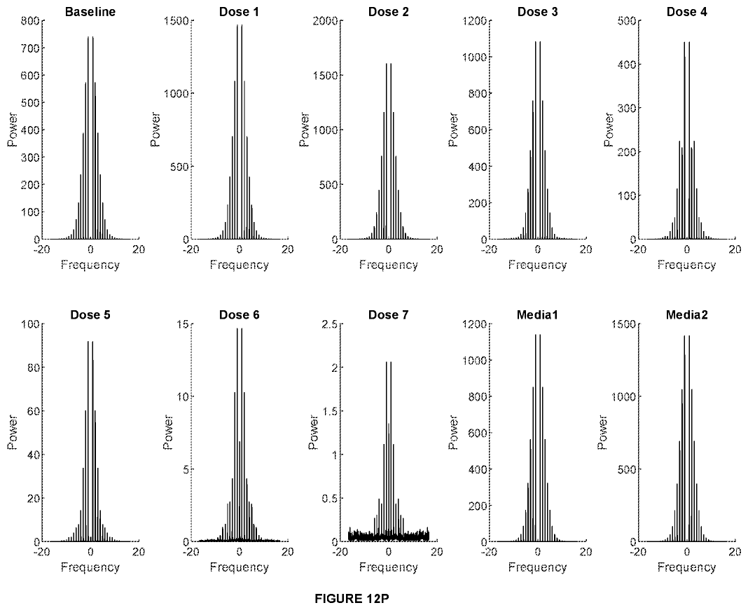

FIGS. 12A-12P show the results of using the systems and methods of the present disclosure to investigate shifts in spectral representations following administration of controls (no drug or dimethyl sulfoxide, DMSO), isoproterenol, or unknown compounds A, B, C, D, E to engineered cardiomyocyte tissues.

In the examples shown in FIGS. 12A-12P, eight artificial adult cardiomyocyte tissues were grown within respective devices of a bioreactor (as described above in relation to FIG. 1A). Once the artificial tissues were matured, each artificial tissue was randomly assigned to one of the following treatments: no drug; DMSO; isoproterenol; unknown compound A; unknown compound B; unknown compound C; unknown compound D; or unknown compound E. Each artificial tissue then followed the same treatment protocol. First, the artificial tissues were stimulated (paced) at a frequency of 1 Hz for 30 s without any administration of the relevant treatment or agent. The waveforms comprising the functional responses of the artificial tissues over the 30 s periods were then recorded. This was repeated three times to obtain three baseline readings (referred to as “Baseline”, “Media1”, and “Media2” in FIGS. 12A-12P). Repeating the baseline measurement helps the artificial tissues to have a fixed stabilization. After stabilization and the recordal of the baseline waveforms, a first dose of the treatment was given to each artificial tissue. The tissues were again stimulated at 1 Hz for 30 s and the resulting waveforms recorded (“Dose 1” in FIGS. 12A-12P). A second dose, greater than the first dose, was then administered to the artificial tissues and the stimulation and recordal process repeated (“Dose 2” in FIGS. 12A-12P). The dosages given to the artificial tissues were sequentially increased in this way with the relevant waveforms recorded up until the seventh dose (“Dose 7” in FIGS. 12A-12P).

FIG. 12A shows example waveforms obtained for the artificial cardiomyocyte tissues.

The waveforms shown in FIG. 12A correspond to responses of the artificial tissues after administration of the final treatment (i.e., “Dose 7”). As can be seen, the functional responses of the artificial tissues within each of the waveforms differ. However, efficiently and accurately capturing the differences in response directly from the waveforms (e.g., using standard feature extraction approaches) may not sufficiently capture the differences in behavior. Moreover, comparing the waveforms is not a straightforward operation due to the complexity and noise present in the waveform representations. Beneficially, by using the spectral representations described in the present disclosure, the different behaviors of the artificial tissues are efficiently and robustly encoded allowing for direct comparison, grouping, and classification.

FIG. 12B shows spectral representations computed from the waveforms shown in FIG. 12A.

The spectral representations shown in FIG. 12B encode the behaviors of the artificial tissues. For example, the spectral representation for the unknown compound A (“Fo-A”) shows increased power at the harmonics thereby encoding a tissue behavior corresponding to an evoked stable contractile response. Similarly, the spectral representation for the unknown compound D (“Fo-D”) shows the main spectral power at 0 Hz thereby encoding a tissue behavior corresponding to a loss of evoked contractions. This behavior is difficult to observe or capture when considering the waveform representation for unknown compound D shown in FIG. 12A because it is not obvious from the waveform representation whether any evoked contractions are present. Moreover, the waveform for unknown compound D and unknown compound E would appear similar (see FIG. 12A) whilst their corresponding spectral representations help reveal a key difference in behavior-unknown compound D exhibits a loss of evoked contractions whilst unknown compound E exhibits a decrease in force with some loss of evoked contractions. The spectral representations thus help uncover different behaviors which are not readily identifiable within the waveform representations of the contractile responses of the artificial tissues.

FIG. 12C illustrates different behaviors encoded by the spectral signatures shown within the examples of FIGS. 12A-12P.

The six different spectral hallmarks, or signatures, shown in FIG. 12C correspond to different behaviors exhibited by the artificial cardiomyocyte tissues. The example signatures correspond to (in order from top left to bottom right): evoked pre-stabilization; evoked stabilization; evoked abnormal response; increased contraction duration; decreased contractile force; and loss of evoked contractions. Therefore, spectral representations provide a robust representation of tissue behavior which allows for different tissue behaviors to be grouped and classified.

FIG. 12D shows waveforms and corresponding spectral representations for artificial tissues under control conditions.

The examples shown in FIG. 12D correspond to artificial tissues which exhibit normal contractile responses under control conditions (left-hand column) and abnormal contractile responses under control conditions (middle and right-hand column). The normal contractile response corresponds to the sixth dosage of DMSO and the abnormal contractile responses correspond to the sixth and seventh readings (i.e., “doses”) of the no drug treatment. Although the artificial tissue for the no drug treatment is under control, or reference, conditions, it nevertheless exhibits abnormal contractile responses in that some double contractions (or double beats) are present. This behavior is encoded in the shifts seen in the spectral representations.

FIGS. 12E and 12F show the waveforms and corresponding spectral representations for an artificial tissue treated with sequential doses of isoproterenol. As seen in the waveforms corresponding to the response due to higher doses of isoproterenol, abnormal beats are induced (see FIG. 12E). This behavior is exhibited in the spectral representations (FIG. 12F) by the diminishment of harmonic localized power densities.

FIGS. 12G and 12H show the waveforms and corresponding spectral representations for an artificial tissue treated with unknown compound A. The waveforms shown in FIG. 12G comprise evoked contractions with increased amplitude wavelength as well as reduced noise. This is exhibited in the spectral representations (FIG. 12H) as a shift to primary frequency (1 Hz) from harmonics.

FIGS. 121 and 12J show the waveforms and corresponding spectral representations for an artificial tissue treated with unknown compound B. At the higher doses of compound D, a loss of evoked contractions can be seen in the waveforms (FIG. 12I). This results in a shift to very high frequency power, indicating all noise and no periodic signal (see, FIG. 12J). However, of note is that hidden in Dose 4, very small contractions were still present. These contractions are not immediately visible in the raw waveform but become visible in the spectral representation.

FIGS. 12K and 12L show the waveforms and corresponding spectral representations for an artificial tissue treated with unknown compound C. As can be seen when comparing the seventh dose to the baseline response for both the waveform representation and the spectral representation, there is little if any obvious effect associated with unknown compound C.

FIGS. 12M and 12N show the waveforms and corresponding spectral representations for an artificial tissue treated with unknown compound D. As can be seen in the spectral representation for the seventh dose, there is a loss of evoked contraction (as captured by the shift to very high frequency power which indicates all noise and no periodic signal). However, this loss did not occur until the final dose, with hidden small contractions evident in the spectral representation of the penultimate dose. As with the example in FIGS. 121 and 12J, these hidden contractions become evident in the spectral representation.

FIGS. 12O and 12P show the waveforms and corresponding spectral representations for an artificial tissue treated with unknown compound E. As the dosage is increased, there is an induced decrease of contraction force and possible full loss of evoked contractions. Even at the highest dosage where contractions were very small, the spectral representation reveals the hidden contractions which are not visible within the raw waveform. The example shown in relation to Dose 7 of FIGS. 12O and 12P demonstrates that the spectral representation approach of the present disclosure can help uncover hidden, small contractions quickly and robustly.

1.-95. (canceled)

96. A system configured to model tissue behavior, the system comprising:
one or more bioreactors configured for growing a plurality of engineered tissues therein; and
a control unit communicatively coupled to the one or more bioreactors, and comprising one or more processors configured to execute instructions that cause the one or more processors to:
obtain a plurality of waveforms comprising functional responses of the plurality of engineered tissues within the one or more bioreactors over time, the plurality of engineered tissues comprising at least a first tissue group associated with a first behavior and a second tissue group associated with a second behavior;
transform the plurality of waveforms to a corresponding plurality of spectral representations, wherein the corresponding plurality of spectral representations provide frequency-based characterizations of the functional responses of the plurality of engineered tissues;
generate a prediction model using the corresponding plurality of spectral representations, wherein the prediction model is trained to output a probability that one or more tissues exhibit the first behavior or the second behavior based on the plurality of spectral representations;
inputting, into the prediction model, a spectral representation of a tissue;
outputting, by the prediction model, a prediction based on the probability indicating that the tissue exhibits the first behavior or the second behavior; and
detecting, based on the prediction, a behavior of the tissue.

97. The system of claim 96 wherein the behavior of the tissue is associated with the first behavior or the second behavior.

98. The system of claim 96 wherein the first behavior is associated with a first mechanism of action and the second behavior is associated with a second mechanism of action.

99. The system of claim 96 wherein the step of obtaining the first spectral representation comprises:
obtaining, by the one or more processors, a first waveform comprising a functional response of the first tissue over a first time frame; and
transforming, by the one or more processors, the first waveform to the first spectral representation.

100. The system of claim 99 wherein the step of obtaining the first spectral representation comprises:
causing stimulation of the first tissue at a first pacing frequency over the first time frame such that the first waveform comprises a functional response of the first tissue stimulated at the first pacing frequency.

101. The system of claim 100 wherein the first pacing frequency is from 0.1 Hz to 20 Hz.

102. The system of claim 100 wherein, the plurality of waveforms comprise functional responses of the plurality of engineered tissues stimulated at a second pacing frequency.

103. The system of claim 102 wherein the first pacing frequency and the second pacing frequency are the same.

104. The system of claim 96 wherein the first tissue is associated with an unknown behavior.

105. The system of claim 96 wherein the first tissue is an engineered tissue.

106. The system of claim 96 wherein the first tissue group are associated with a first cell line and the second tissue group are associated with a second cell line, wherein the second cell line is different to the first cell line.

107. The system of claim 96 wherein the first tissue group are associated with a first intervention and the second tissue group are associated with a second intervention.

108. The system of claim 96 wherein the prediction model comprises a clustering model.

109. The system of claim 108 wherein the prediction model assigns the probability based on a first distance between the spectral representation of the tissue and a first centroid associated with the first behavior or a second distance between the spectral representation of the tissue and a second centroid associated with the second behavior.

110. The system of claim 109 wherein the first centroid and the second centroid are determined by the clustering model.

111. The system of claim 96 wherein the prediction model comprises a classifier.

112. The system of claim 111 wherein the step of generating the prediction model comprises training the classifier on the corresponding plurality of spectral representations.

113. The system of claim 96 wherein the plurality of engineered tissue comprise engineered muscle tissue and the first tissue comprises muscle tissue.

114. A non-transitory computer-readable medium storing instructions for modeling tissue behavior, the instructions which, when executed by one or more processors, cause the one or more processors to
obtain a plurality of waveforms comprising functional responses of a plurality of engineered tissues within one or more bioreactors over time, the plurality of engineered tissues comprising at least a first tissue group associated with a first behavior and a second tissue group associated with a second behavior;
transform the plurality of waveforms to a corresponding plurality of spectral representations, wherein the corresponding plurality of spectral representations provide frequency-based characterizations of the functional responses of the plurality of engineered tissues;
generate a prediction model using the corresponding plurality of spectral representations, wherein the prediction model is trained to output a probability that one or more tissues exhibit the first behavior or the second behavior based on the plurality of engineered tissues;
input into the prediction model, a spectral representation of a tissue;
output, by the prediction model, a prediction based on the probability indicating that the tissue exhibits the first behavior or the second behavior; and
detect, based on the prediction, a behavior of the tissue.

115. A computer-implemented method for modeling tissue behavior, the method comprising:
obtaining, by one or more processors, a plurality of waveforms comprising functional responses of a plurality of engineered tissues within one or more bioreactors over time, the plurality of engineered tissues comprising at least a first tissue group associated with a first behavior and a second tissue group associated with a second behavior;
transforming, by the one or more processors, the plurality of waveforms to a corresponding plurality of spectral representations, wherein the corresponding plurality of spectral representations provide frequency-based characterizations of the functional responses of the plurality of engineered tissues;
generating, by the one or more processors, a prediction model using the corresponding plurality of spectral representations, wherein the prediction model is trained to output a probability that one or more tissues exhibit the first behavior or the second behavior based on the plurality of engineered tissues;
inputting into the prediction model, a spectral representation of a tissue;
outputting, by the prediction model, a prediction based on the probability indicating that the tissue exhibits the first behavior or the second behavior; and
detecting, based on the prediction, a behavior of the tissue.
